(19) United States
(12) Patent Application Publication
(10) Pub. No.: US 2025/0236879 A1
(43) Pub. Date: Jul. 24, 2025

(54) PLANT REGULATORY ELEMENTS AND USES THEREOF

(71) Applicant: The Board of Trustees of the University of Illinois, Urbana, IL (US)

(72) Inventors: Laurie Leonelli, Champaign, IL (US); Jake Harris, Champaign, IL (US); Jooyeon Jeong, Champaign, IL (US)

(21) Appl. No.: 19/033,029

(22) Filed: Jan. 21, 2025

Related U.S. Application Data
(60) Provisional application No. 63/623,664, filed on Jan. 22, 2024.

(51) Int. Cl. C12N 15/82

(52) U.S. Cl. CPC C12N 15/8261; C12N 15/8205; C12N 15/8273

(57) ABSTRACT

The present invention provides novel DNA molecules and constructs, including their nucleotide sequences, useful for modulating gene expression in plants and plant cells. The invention also provides transgenic plants, plant cells, plant parts, seeds, and commodity products comprising the DNA molecules operably linked to heterologous transcribable polynucleotides, along with methods of their use. The invention also provides methods for identifying regulatory elements.

### CROSS-REFERENCE TO RELATED APPLICATIONS

This application claims priority to U.S. Provisional Application Ser. No. 63/623,664 filed on Jan. 22, 2024, the entire disclosure of which is incorporated herein by reference.

### INCORPORATION OF SEQUENCE LISTING

The sequence listing that is contained in the file named “AGOE015US_ST26.xml,” which is 23.6 KB (as measured in Microsoft Windows®) and was created on Jan. 21, 2025, is filed herewith by electronic submission, and is incorporated by reference herein.

### FIELD OF THE INVENTION

The invention relates to the field of plant molecular biology and plant genetic engineering, and DNA molecules useful for modulating gene expression in plants.

### BACKGROUND

Regulatory elements are genetic elements that regulate gene activity by modulating the transcription of an operably linked transcribable polynucleotide molecule. Such elements include promoters, leaders, introns, and 3′ untranslated regions and are useful in the field of plant molecular biology and plant genetic engineering.

### SUMMARY OF THE INVENTION

The present invention provides novel gene regulatory elements for use in plants. The present invention also provides DNA constructs comprising the regulatory elements. The present invention also provides transgenic plant cells, plants, and seeds comprising the regulatory elements. The sequences may be provided operably linked to a transcribable polynucleotide molecule. In one embodiment, the transcribable polynucleotide molecule may be heterologous with respect to a regulatory sequence provided herein. A regulatory element sequence provided by the invention thus may, in particular embodiments, be defined as operably linked to a heterologous transcribable polynucleotide molecule. The present invention also provides methods of making and using the regulatory elements, the DNA constructs comprising the regulatory elements, and the transgenic plant cells, plants, and seeds comprising the regulatory elements operably linked to a transcribable polynucleotide molecule.

Thus, in one aspect, the present invention provides a DNA molecule comprising a DNA sequence selected from the group consisting of: a) a sequence with at least about 85 percent sequence identity to any of SEQ ID NOs:1-8; b) a sequence comprising any of SEQ ID NOs:1-8; c) a fragment of a sequence having at least 85 percent sequence identity to any of SEQ ID NOs:1-8, wherein the fragment has gene-regulatory activity; d) a fragment of any of SEQ ID NOs:1-8, wherein the fragment has gene-regulatory activity; and c) combinations thereof; wherein the sequence is operably linked to a heterologous transcribable polynucleotide molecule. In some embodiments, the DNA molecule is active as a promoter. In further embodiments, the DNA molecule further comprises a heterologous regulatory clement. In specific embodiments, the DNA molecule comprises at least about 90 percent, at least about 95 percent, at least about 98 percent, or at least about 99 percent sequence identity to the DNA sequence of any of SEQ ID NOs:1-8. In certain embodiments of the DNA molecule, the DNA sequence comprises a regulatory element. In some embodiments, the regulatory element comprises a promoter. In particular embodiments, the heterologous transcribable polynucleotide molecule comprises a gene of agronomic interest, such as a gene capable of providing increased yield in plants, a gene capable of providing increased root growth in plants, a gene capable of providing increased drought resistance in plants, or a gene capable of providing increased starch content in plants.

In another aspect, the invention provides a construct comprising at least one copy of a DNA molecule provided herein, and an operably linked transcribable gene of agronomic interest. In some embodiments, the construct comprises in the 5′-3′ direction: (a) the at least one copy of said DNA molecule; (b) the operably linked transcribable gene of agronomic interest; and (c) a gene termination sequence. In further embodiments, the transcribable gene of agronomic interest comprises an open reading frame encoding a polypeptide.

The invention also provides a transgenic plant cell comprising a heterologous DNA construct provided by the invention, including a sequence of any of SEQ ID NOs: 1-8, or a fragment or variant thereof, wherein said sequence is operably linked to a heterologous transcribable polynucleotide molecule. For example, in further embodiments, the transgenic plant cell may comprise a sequence selected from the group consisting of: a) a sequence with at least 85 percent sequence identity to any of SEQ ID NOs:1-8; b) a sequence comprising any of SEQ ID NOs:1-8; c) a fragment of a sequence having at least 85 percent sequence identity to any of SEQ ID NOs:1-8,wherein the fragment has gene-regulatory activity; d) a fragment of any of SEQ ID NOs:1-8,wherein the fragment has gene-regulatory activity; and e) combinations thereof; wherein said sequence is operably linked to a heterologous transcribable polynucleotide molecule. In certain embodiments, the transgenic plant cell is a monocotyledonous plant cell. In other embodiments, the transgenic plant cell is a dicotyledonous plant cell. In particular embodiments, the transgenic plant cell is a cassava plant cell.

Further provided by the invention is a transgenic plant, or part thereof, comprising a DNA molecule as provided herein, including a DNA sequence selected from the group consisting of: a) a sequence with at least 85 percent sequence identity to any of SEQ ID NOs:1-8; b) a sequence comprising any of SEQ ID NOs:1-8; c) a fragment of a sequence having at least 85 percent sequence identity to any of SEQ ID NOs:1-8, wherein the fragment has gene-regulatory activity; d) a fragment of any of SEQ ID NOs:1-8, wherein the fragment has gene-regulatory activity; and c) combinations thereof; wherein said sequence is operably linked to a second heterologous transcribable polynucleotide molecule. In specific embodiments, the transgenic plant may be a progeny plant of any generation that comprises the DNA molecule, relative to a starting transgenic plant comprising the DNA molecule. Still further provided is a transgenic seed comprising a DNA molecule according to the invention.

In yet another aspect, the invention provides a method of producing a commodity product comprising obtaining a transgenic plant or part thereof according to the invention and producing the commodity product therefrom. In one embodiment, a commodity product of the invention is protein concentrate, protein isolate, grain, starch, seeds, meal, flour, biomass, or seed oil. In another aspect, the invention provides a commodity produced using the above method. For instance, in one embodiment the invention provides a commodity product comprising a DNA molecule as provided herein, including a DNA sequence selected from the group consisting of: a) a sequence with at least 85 percent sequence identity to any of SEQ ID NOs:1-8; b) a sequence comprising any of SEQ ID NOs:1-8; c) a fragment of a sequence having at least 85 percent sequence identity to any of SEQ ID NOs:1-8, wherein the fragment has gene-regulatory activity; d) a fragment of any of SEQ ID NOs:1-8, wherein the fragment has gene-regulatory activity; and c) combinations thereof; wherein the sequence is operably linked to a heterologous transcribable polynucleotide molecule.

In still yet another aspect, the invention provides a method of expressing a transcribable polynucleotide molecule that comprises obtaining a transgenic plant according to the invention, such as a plant comprising a DNA molecule as described herein, and cultivating a plant, wherein a transcribable polynucleotide in the DNA molecule is expressed.

In a further aspect, methods of in planta transient agrobacterium infiltration (agroinfiltration) are provided. In certain embodiments, an Agrobacterium tumefaciens strain such as AGL1, C58C1, EHA105, or GV3101 is transformed with a genetic construct as described herein, preferably strain AGLI. Recipient plants are grown to various vegetative stages before infiltration, preferably early vegetative stages between about 2 weeks to about 5 weeks. Recipient plants may be any plant, such as in certain embodiments a legume plant, for example a cowpea plant. Agrobacterium inoculum for infiltration may be at any concentration, preferably between an OD₆₀₀ of about 0.1 to about 0.8, most preferably between an OD₆₀₀ of about 0.3 to about 0.5. In certain embodiments, infiltration of the youngest fully expanded trifoliate leaves are preferred, such as fully expanded trifoliate leaves between about 2 weeks to about 5 weeks, preferably about 2 weeks. In certain examples, gene expression in transiently transformed leaves may be evaluated in leaf punches, for example using a detectable marker.

Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

FIG. 1 shows efficiency of Agrobacterium strain and OD on transient expression in cowpea. Panel (a): 2-weck-old cowpea with the first fully-expanded trifoliate used for agroinfiltration. Panel (b): Betalain accumulating in cowpea inoculated with four different agrobacterium strains. Panel (c): Betalain absorbance in leaves infiltrated with different agrobacterium strains. Panel (d): Betalain absorbance in leaves inoculated with different concentrations of AGLI strain. Absorbance data were normalized by dividing each absorbance value by the sum of absorbances from individual leaf replicates to control for variation between leaves and statistical significance was determined by One-way ANOVA followed by pairwise t-tests (n=11). Error bar represents SD.

FIG. 2 shows a transient gene expression assay using cowpea at various carly vegetative stages. Panel (a): Cowpea plants at vegetative stages ranging from 2 weeks to 5 weeks old. Green leaves indicate youngest fully-expanded leaves. Panel (b): Betalain absorbance measured from all trifoliate leaves. Statistical significance was determined by One-way ANOVA followed by pairwise t-tests and Kruskal-Wallis test followed by Dunn's test (n=8).

FIG. 3 shows identification of constitutive promoters in cowpea. Panel (a): Native gene expression driven by six candidate promoters (VuCP01 to VuCP06) identified from our cowpea RNA-seq expression data across all canopy layers (top, middle, and bottom) and developmental stages (R1 and R3). Panel (b): Betalain accumulating pattern and absorbance in Nicotinana benthamiana leaves expressing RUBY constructs driven by the candidate promoters (n=4). Panel (c): Betalain accumulating pattern and absorbance in cowpea leaves expressing RUBY constructs driven by the candidate promoters (n=8). Error bar represents SD.

FIG. 4 shows characterization of drought-specific promoters. Panel (a): Stomatal conductance over the course of drought treatment. Panel (b): Betalain accumulation in leaves during drought stress. Panel (c): Betalain absorbance of fast drought-treated cowpea leaves expressing VuDP01-and VuDP02-driven RUBY constructs 96 hours after the treatment. Error bar represents SD (n=18). Statistical significances were analyzed by Mann-Whitney test (a; *p<0.001) and Kruskal-Wallis test followed by Dunn's test (c).

FIG. 5 shows cowpea gene expression plots for exemplary cowpea gene Vigun01g082900 at different canopy positions and developmental stages displayed in the CowPEAsy Shiny application. Starting from the full set of genes in the RNA-seq dataset, the DESeq2 package will filter out genes with zero or very low read counts and genes with extreme outliers. Following this, user-defined adjusted p-value and baseMean (average normalized counts across all canopy positions and development stages) are used to further filter the dataset. Finally, user-defined mean expression ranges are used to filter for desired canopy expression profiles.

FIG. 6 shows induction of fast and slow drought stresses to the cowpea plants infiltrated with constructs containing 35S, VuDP01, and VuDP02 promoters, respectively, linked to the RUBY reporter. Stomatal conductance, panel (a); operating photosystem II efficiency, panel (b); and leaf images, panel (c); were monitored throughout the treatment. Error bars represent SD (n=3 for slow stress; n=6 for fast stress and well-watered control). Statistical significances against to well-watered control were analyzed by Kruskal-Wallis test followed by Dunn's test with Benjamini-Hochberg correction (*p<0.05; **p<0.01).

SEQ ID NO: 7 shows expression patterns during drought stress in cowpea infiltrated with constructs containing either the VuDP01 (Panel A) or VuDP02 (Panel B) promoters to express the RUBY reporter. Fold changes in VuDREB2A, VuCPRD22, and RUBY were calculated by normalizing the relative expression levels to the reference genes (VuRPL40e and VuUE21D), and to the 0-hour time point. Error bars represent 95% confidence interval (n=3). Statistical significance was determined by one-way ANOVA followed by pairwise t-tests comparing each time point to the 0-hour time point (*p<0.05; **p<0.01; ***p<0.001).

### BRIEF DESCRIPTION OF THE SEQUENCES

SEQ ID NO: 1 is promoter sequence VuCPO1. identified upstream of Vigna unguiculata Gene ID Vigun01g219300.

SEQ ID NO: 2 is promoter sequence VuCPO2, identified upstream of Vigna unguiculata Gene ID Vigun11g163500.

SEQ ID NO: 3 is promoter sequence VuCPO3, identified upstream of Vigna unguiculata Gene ID Vigun04g096500.

SEQ ID NO: 4 is promoter sequence VuCPO4, identified upstream of Vigna unguiculata Gene ID Vigun08g150700.

SEQ ID NO: 5 is promoter sequence VuCPO5, identified upstream of Vigna unguiculata Gene ID Vigun09g108850.

SEQ ID NO: 6 is promoter sequence VuCPO6, identified upstream of Vigna unguiculata Gene ID Vigun02g039700.

SEQ ID NO: 7 is promoter sequence VuDP01, identified upstream of Vigna unguiculata Gene ID Vigun04g020700.

SEQ ID NO: 8 is promoter sequence VuDP02, identified upstream of Vigna unguiculata Gene ID Vigun08g165400.

### DETAILED DESCRIPTION OF THE INVENTION

There is an urgent need to increase agricultural output in Sub-Saharan Africa to combat hunger and malnutrition. According to the latest Food and Agricultural Organization of the United Nations (FAO) report, 239 million people in Sub-Saharan Africa suffer from chronic hunger; and 399 million people are at least moderately food insecure. Furthermore, the food insecurity situation in Sub-Saharan Africa has grown worse in recent years, due in part to changing climate, conflict, and economic slowdowns.

Cowpea (Vigna unguiculata L. Walp) is a grain legume crop that is widely cultivated in over 60 countries. Since cowpea is resilient to harsh growing conditions, such as drought and low soil fertility, it is one of the staple crops in Sub-Saharan Africa. It is an important source of essential dietary nutrients for human consumption and livestock feed due to providing a high proportion of protein. It is also a significant source of income for smallholder farmers, as it can be grown in rotation with major cereal crops and can be used as a cover crop to improve soil fertility.

Despite its importance, an average yield of cowpea in Africa is between 100 to 600 kg/ha, which is far below its potential yield of 1500 to 3000 kg/ha. Biotic stresses such as parasitic weeds, insect pests, Root-Knot Nematodes, and diseases caused by fungi, viruses, and bacteria have been identified as constraints on cowpea production. Additionally, abiotic stresses such as drought, heat, and waterlogging due to climate change, also contribute to low photosynthetic rate and biomass of cowpea. Traditional breeding programs have made some progress in addressing these challenges, but these approaches are limited by existing genetic backgrounds and require several rounds of backcrossing to breed out undesirable traits that result from the initial cross.

Maximizing productivity through genetic engineering in cowpea has been slowed due to a lack of defined species-specific regulatory elements and an inability to test gene function in the native system. In other plant species, Agrobacterium-mediated transient gene expression is widely used to validate constructs before investing in transgenic lines, but its adaptation to legumes has been challenging. The present inventors have therefore developed a betalain reporter to optimize an in planta agroinfiltration transient assay in trifoliate cowpea leaves. This assay was then used to characterize six cowpea promoters identified through transcriptomics that show constitutive expression across three canopy positions and two developmental time points, effectively creating a suite of physiologically relevant promoters of varied strengths. The “intact plant” aspect of the transient system has allowed for characterization of drought-inducible promoters by challenging cowpea plants with drought stress. These findings provide an in vivo platform for validation of cis-regulatory elements in cowpea and other legumes and enhance current genetic resources by providing RNA sequencing data from different canopy levels and developmental stages in cowpea.

The present disclosure further provides novel, drought-inducible gene-regulatory polynucleotides developed using RNA-seq data and validated using the agroinfiltration method disclosed herein. In certain embodiments promoter sequences such as SEQ ID NOs:1-8 are provided for regulation of transgene expression in plants, including cowpea. These polynucleotide molecules are, for instance, capable of affecting the expression of an operably linked transcribable polynucleotide molecule in plant tissues, and therefore selectively regulating gene expression, or activity of an encoded gene product, in transgenic plants. The present invention also provides methods of modifying, producing, and using the same. The invention also provides compositions, transformed host cells, transgenic plants, and seeds containing the promoters and/or other disclosed nucleotide sequences, and methods for preparing and using the same.

The present disclosure results from a comprehensive examination of gene expression across layers of canopy and developmental stages. This interactive database has the potential to relieve challenges to canopy engineering by facilitating identification of canopy-specific promoters to differentially express target genes at each canopy level. Given the limited number of regulatory elements currently confirmed in cowpea, the constitutive promoters identified and validated herein are promising candidates for achieving constitutive expression of genes of interest at different intensities or levels throughout the plant.

The agroinfiltration method described herein was further used to test drought-inducible candidate promoters by subjecting the whole plants to drought stress following the transient assay. Considering that drought is a primary abiotic stress with substantial repercussions on cowpea yield, especially in the dry environments where cowpea is typically cultivated, a further understanding of drought response in cowpea and enhancing its drought resilience are of great importance. The drought-inducible promoter sequences provided herein will therefore allow for more effective expression of transgenes in response to environmental conditions.

The following definitions and methods are provided to better define the present invention and to guide those of ordinary skill in the art in the practice of the present invention. Unless otherwise noted, terms are to be understood according to conventional usage by those of ordinary skill in the relevant art.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used hercin, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, or a DNA molecule that has been incorporated into a host cell's DNA by genetic transformation or gene editing.

The polynucleotides disclosed herein may be synthetic nucleotide sequences. A “synthetic nucleotide sequence” is a nucleotide sequence that is not known to occur in nature or that is not naturally occurring. In some embodiments, the polynucleotide shares little or no extended homology to natural sequences. Extended homology in this context generally refers to 100% sequence identity extending beyond about 25 nucleotides of contiguous sequence.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragments thereof, can also be obtained by other techniques such as by directly synthesizing the fragment by chemical means, as is commonly practiced by using an automated oligonucleotide synthesizer.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” refers to a sequence provided as the polynucleotide sequences of SEQ ID NOs:1-8.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a DNA molecule comprising a sequence that when optimally aligned to a reference sequence, provided herein as SEQ ID NOs:1-8, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments, such sequences may be defined as having gene-regulatory activity or having the activity of the reference sequence.

### Regulatory Elements

A regulatory element is a DNA molecule having gene regulatory activity, i.e., one that has the ability to affect the transcription and/or translation of an operably linked transcribable polynucleotide molecule. Regulatory clements or promoter elements described herein include polynucleotides having a sequence of any of SEQ ID NOs:1-8. The term “gene regulatory activity” thus refers to the ability to affect the expression pattern of an operably linked transcribable polynucleotide molecule by affecting the transcription and/or translation of that operably linked transcribable polynucleotide molecule. As used herein, a transcriptional regulatory sequence may be comprised of operably linked expression elements, such as enhancers, promoters, leaders, such as 5′-untranslated regions or part thereof, introns, 3′-untranslated regions or part thereof, terminators, transcription termination regions (or 3′ UTRs), or chromatin control elements that function in plants can therefore be useful for modifying plant phenotypes through genetic engineering. Thus, a transcriptional regulatory sequence may be comprised, for instance, of a promoter operably linked 5′ to a leader sequence, which is in turn operably linked 5′ to an intron sequence. Leaders and introns may positively affect transcription of an operably linked transcribable polynucleotide molecule as well as translation of the resulting transcribed RNA. The pre-processed RNA molecule comprises leaders and introns, which may affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule.

Regulatory elements such as promoters, enhancers, leaders, such as 5′-untranslated regions or part thereof, introns, 3′-untranslated regions or part thereof, transcription termination regions (or 3′ UTRs), or chromatin control elements are DNA molecules that have gene regulatory activity and play an integral part in the overall expression of genes in living cells. The term “regulatory element” refers to a DNA molecule having gene regulatory activity, i.e., one that has the ability to affect the transcription and/or translation of an operably linked transcribable polynucleotide molecule. Isolated regulatory elements, such as promoters and leaders that function in plants are therefore useful for modifying plant phenotypes through the methods of genetic engineering.

It is recognized that polynucleotides of the present invention can comprise a plurality of regulatory elements such as, for example, a promoter and an enhancer. It is further recognized that some genetic regulatory elements act in concert with other genetic regulatory elements to control the regulation of an operably linked gene of interest. Moreover, it is recognized that some genetic regulatory elements such as, for example, a promoter or enhancer, can be separated from the transcribed region of a gene of interest by 1, 2, 3, or more kilobases of DNA.

The present invention also provides methods for controlling gene expression. “Controlling gene expression” refers to controlling the expression of an RNA transcript, and can further encompass translation of the transcript, or even an activity or function of the encoded protein. Controlling gene expression can include affecting one or more of RNA transcription, processing, turnover, and/or translation.

The genetic regulatory elements as disclosed herein can be implemented as regulatory sequences to control gene expression in a “desired manner.” The desired manner of gene expression can be temporally, spatially, or any combination thereof in a target organism including, but not limited to, constitutive expression, tissue-preferred expression, and organ-preferred expression. The desired manner of gene expression can also be expression in response to biotic stress (e.g., fungal, bacterial, and viral pathogens, insects, herbivores, and the like) and/or abiotic stress (c.g., wounding, drought, cold, heat, high nutrient levels, low nutrient levels, metals, light, herbicides and other synthetic chemicals, and the like).

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a dsRNA, a tRNA, an rRNA, a miRNA, and the like.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

### Promoters

A regulatory clement, such as a promoter of the invention including promoters of SEQ ID NOs:1-8, may be operably linked to a transcribable DNA molecule that is heterologous with respect to the regulatory element. As used herein, the term “heterologous” refers to the combination of two or more DNA molecules when such a combination is not normally found in nature. For example, the two DNA molecules may be derived from different species, and/or the two DNA molecules may be derived from different genes, e.g., different genes from the same species or the same genes from different species. A regulatory element is thus heterologous with respect to an operably linked transcribable DNA molecule if such a combination is not normally found in nature, i.e., the transcribable DNA molecule does not naturally occur operably linked to the regulatory element.

The transcribable DNA molecule may generally be any DNA molecule for which expression of a transcript is desired. Such expression of a transcript may result in translation of the resulting mRNA molecule, and thus protein expression. Alternatively, for example, a transcribable DNA molecule may be designed to ultimately cause decreased expression of a specific gene or protein. In one embodiment, this may be accomplished by using a transcribable DNA molecule that is oriented in the antisense direction. One of ordinary skill in the art is familiar with using such antisense technology. Any gene may be negatively regulated in this manner, and, in one embodiment, a transcribable DNA molecule may be designed for suppression of a specific gene through expression of a dsRNA, siRNA, or miRNA molecule.

In some embodiments, the present disclosure provides polynucleotides, including polynucleotides having the sequence of SEQ ID NO:1-8, containing promoters and/or enhancers. “Promoter” refers to a nucleotide sequence that is capable of controlling the expression of an operably linked coding sequence or other sequence encoding an RNA that is not necessarily translated into a protein. Thus, the polynucleotide may comprise proximal promoter elements as well as more distal upstream elements, the latter elements often referred to as enhancers. An “enhancer” refers to a DNA sequence that can stimulate promoter activity and may be an innate element of the promoter or a heterologous element inserted to enhance the level or tissue-specificity of a promoter. It is understood by those skilled in the art that different promoters may direct the expression of a gene in different tissues or cell types, at different stages of development, or in response to different environmental conditions. It is further recognized that because in most cases the exact boundaries of regulatory sequences have not been completely defined, nucleic acid fragments of some variation may have identical or similar promoter activity.

Promoters that cause a gene to be expressed in most cell types of an organism and at most times are commonly referred to as “constitutive promoters.” Expression of a gene in most cell types of an organism and at most times is referred to herein as “constitutive gene expression” or “constitutive expression.”

In some embodiments, a regulatory element is an expression-enhancing intron. An “expression-enhancing intron” or “enhancing intron” is an intron that is capable of causing an increase in the expression of a gene to which it is operably linked. While the present invention is not known to depend on a particular biological mechanism, it is believed that the expression-enhancing introns of the present invention enhance expression through intron mediated enhancement (IME). It is recognized that naturally occurring introns that enhance expression through IME are typically found within 1 Kb of the transcription start site of their native genes (see, Rose et al. (2008) Plant Cell 20:543-551). Such introns are usually the first intron, whether the first intron is in the 5′ UTR or the coding sequence and need to be in a transcribed region. Introns that enhance expression solely through IME do not enhance gene expression when they are inserted into a non-transcribed region of a gene, such as, for example, a promoter. That is, they do not function as transcriptional enhancers. Unless stated otherwise or apparent from the context, the expression-enhancing introns of the present invention are capable of enhancing gene expression when they are found in a transcribed region of a gene but not when they occur in a non-transcribed region such as, for example, a promoter.

In some embodiments, the promoter is a plant promoter. A “plant promoter” is a promoter capable of initiating transcription in plant cells whether or not its origin is a plant cell, e.g., it is well known that Agrobacterium promoters are functional in plant cells. Thus, plant promoters include promoter DNA obtained from plants, plant viruses and bacteria such as Agrobacterium and Bradyrhizobium bacteria, and synthetic promoters capable of initiating transcription in plant cells. A plant promoter can be a constitutive promoter, a non-constitutive promoter, an inducible promoter, a repressible promoter, a tissue specific promoter (e.g., a root specific promoter, a stem specific promoter, a leaf specific promoter), a tissue preferred promoter (e.g., a root preferred promoter, a stem preferred promoter, a leaf preferred promoter), a cell type specific or preferred promoter (e.g., a meristem cell specific/preferred promoter), or many other types. In some embodiments, the variant polynucleotides or fragments described herein include additional known cis-acting sequences to drive expression of a transcribed gene in a desired manner.

In some embodiments, the promoter is a constitutive promoter. A “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. For illustration, constitutive promoters can include CaMV 19S promoter, CaMV 35S promoter (U.S. Pat. Nos. 5,352,605; 5,530,196 and 5,858,742), opine promoters, ubiquitin promoter, actin promoter, alcohol dehydrogenase promoter, etc. In some embodiments, the synthetic promoter prepared as described herein, is used to drive expression of a heterologous sequence, while CaMV 35S promoter is used to drive expression of a second sequence.

In some embodiments, the promoter is a non-constitutive promoter. A “non-constitutive promoter” is a promoter which is active under certain conditions, in certain types of cells, and/or during certain development stages. For example, tissue specific, tissue preferred, cell type specific, cell type preferred, inducible promoters, and promoters under developmental control are non-constitutive promoters. Examples of promoters under developmental control include promoters that preferentially initiate transcription in certain tissues, such as stems, leaves, roots, or seeds.

In some embodiments, the promoter is an inducible or a repressible promoter. An “inducible” or “repressible” promoter is a promoter which is under chemical or environmental factor control. Examples of environmental conditions that may affect transcription by inducible promoters include drought, cold, heat, certain chemicals, or the presence of light.

In some embodiments, the promoter is a tissue-specific promoter. A “tissue-specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large number of tissue-specific promoters isolated from particular plants and tissues found in both scientific and patent literature. Non-limiting examples of known tissue-specific promoters can include beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression), and embryo specific promoters, e.g., a promoter associated with an amino acid permease gene (AAP1), an oleate 12-hydroxylase:desaturase gene from Lesquerella fendleri (LFAH12), an 2S2 albumin gene (2S2), a fatty acid clongase gene (FAE1), or a leafy cotyledon gene (LEC2).

In some embodiments, the promoter is a tissue-preferred promoter. A “tissue-preferred” promoter is a promoter that initiates transcription mostly, but not necessarily entirely or solely, in certain tissues.

In some embodiments, the promoter is a cell type-specific promoter. A “cell type specific” promoter is a promoter that primarily drives expression in certain cell types in one or more organs, for example, vascular cells in roots, leaves, stalk cells, and stem cells.

In some embodiments, the promoter is a cell type-preferred promoter. A “cell type preferred” promoter is a promoter that primarily drives expression mostly, but not necessarily entirely or solely in certain cell types in one or more organs, for example, vascular cells in roots, leaves, stalk cells, and stem cells.

In some embodiments, the promoter is a root-specific promoter. A “root-specific” promoter is a promoter that initiates transcription only in root tissues.

In some embodiments, the promoter is a root-preferred promoter. A “root-preferred” promoter is a promoter that initiates transcription mostly, but not necessarily entirely or solely in root tissues.

In some embodiments, the present invention provides for methods to obtain inbred plants comprising the polynucleotide sequences. As used herein, the term “inbred” or “inbred plant” is used in the context of the present invention. This also includes any single gene conversions of that inbred. The phrase “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein, the term “promoter” or a molecule having “promoter activity” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include SEQ ID NO:1-8, or fragments, variants, functional fragments, or variants thereof, or combinations thereof. In specific embodiments of the invention, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription.

In one embodiment, fragments are provided of a promoter sequence disclosed herein. Promoter fragments may comprise promoter activity, as described above, and may be useful alone or in combination with other promoters and promoter fragments, such as in constructing chimeric promoters. In specific embodiments, fragments of a promoter are provided comprising at least about 50, 95, 150, 250, 500, 750, 1000, 1250, 1500, 1750, or at least about 2000 contiguous nucleotides, or longer, of any of SEQ ID NOs:1-8 or a polynucleotide molecule having promoter activity disclosed herein. Fragments of SEQ ID NOs:1-8 may have the activity of the reference promoter sequence.

Compositions derived from any of the promoters presented as SEQ ID NO:1-8, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue or cell specific effects on expression. Compositions derived from any of the promoters presented as SEQ ID NO:1-8 comprised of 3′ deletions in which the TATA box element or equivalent sequence thereof and downstream sequence is removed can be used, for example, to make enhancer elements. Further deletions can be made to remove any elements that have positive or negative; tissue specific; cell specific; or timing specific (such as, but not limited to, circadian rhythms) effects on expression. Any of the promoters presented as SEQ ID NO:1-8 and fragments or enhancers derived therefrom can be used to make chimeric transcriptional regulatory element compositions comprised of any of the promoters presented as SEQ ID NO:1-8 and the fragments or enhancers derived therefrom operably linked to other enhancers and promoters. The efficacy of the modifications, duplications, or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. Leaders known in the art may be useful in practicing the present invention. The leader sequences (5′ UTR) may be comprised of regulatory elements or may adopt secondary structures that can have an effect on transcription or translation of a transgene. Leader sequences known in the art can be used in accordance with the invention to make chimeric regulatory elements that affect transcription or translation of a transgene. In addition, leader sequences can be used to make chimeric leader sequences that affect transcription or translation of a transgene.

In accordance with the invention, a promoter or promoter fragment may be analyzed for the presence of known promoter elements, i.e., DNA sequence characteristics, such as a TATA-box and other known transcription factor binding site motifs. Identification of such known promoter clements may be used by one of skill in the art to design variants of the promoter having a similar expression pattern to the original promoter.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box or equivalent sequence. A promoter may naturally comprise one or more enhancer elements that affect the transcription of an operably linked polynucleotide sequence. An isolated enhancer element may also be fused to a promoter to produce a chimeric promoter cis-element, which confers an aspect of the overall modulation of gene expression. A promoter or promoter fragment may comprise one or more enhancer elements that affect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention. Enhancer sequences derived from the CaMV can also be utilized (U.S. Pat. Nos. 5,164,316; 5,196,525; 5,322,938; 5,530,196; 5,352,605; 5,359,142; and 5,858,742 for example).

In plants, the inclusion of some introns in gene constructs leads to increased mRNA and protein accumulation relative to constructs lacking the intron.

This effect has been termed “intron mediated enhancement” (IME) of gene expression. Introns known to stimulate expression in plants have been identified in maize genes (e.g., tubA1, Adh1, Sh1, Ubi1) and in rice genes (e.g., salt, tpi). Similarly, introns from dicotyledonous plant genes like those from petunia (e.g., rbcS), potato (e.g., st-ls1), and Arabidopsis thaliana (e.g., ubq3 and pat1) have been found to elevate gene expression rates. It has been shown that deletions or mutations within the splice sites of an intron reduce gene expression, indicating that in some cases splicing might be needed for IME.

Enhancement of gene expression by introns is not a general phenomenon because some intron insertions into recombinant expression cassettes fail to enhance expression (e.g. introns from dicot genes (rbcS gene from pea, phascolin gene from bean and the stls-1 gene from Solanum tuberosum) and introns from maize genes (adh1 gene the ninth intron, hsp81 gene the first intron)). Therefore, not every intron can be employed to manipulate the gene expression level of non-endogenous genes or endogenous genes in transgenic plants. What characteristics or specific sequence features must be present in an intron sequence in order to enhance the expression rate of a given gene is not known in the prior art and therefore from the prior art it is not possible to predict whether a given plant intron, when used heterologously, will cause enhancement of expression at the DNA level or at the transcript level (IME).

As used herein, the term “chimeric” refers to a single DNA molecule produced by fusing a first DNA molecule to a second DNA molecule, where neither first nor second DNA molecule would normally be found in that configuration, i.e., fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present invention.

As used herein, the term “variant” refers to a second DNA molecule that is in composition similar, but not identical to, a first DNA molecule and yet the second DNA molecule still maintains the general functionality, i.e., same, or similar expression pattern, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion, and/or insertion of one or more nucleotides of a reference sequence, wherein the derivative regulatory element has more or less or equivalent transcriptional or translational activity than the corresponding parent regulatory molecule. The regulatory element “variants” will also encompass variants arising from mutations that naturally occur in bacterial and plant cell transformation. In the present invention, a polynucleotide sequence provided as SEQ ID NOs:1-8 may be used to create variants that are in composition similar, but not identical to, the polynucleotide sequence of the original regulatory element, while still maintaining the general functionality, i.e. same or similar expression pattern, of the original regulatory element. Production of such variants of the present invention is well within the ordinary skill of the art in light of the disclosure and is encompassed within the scope of the present invention. Chimeric regulatory element “variants” comprise the same constituent elements as a reference sequence but the constituent elements comprising the chimeric regulatory element may be operatively linked by various methods known in the art, such as, restriction enzyme digestion and ligation, ligation independent cloning, modular assembly of PCR products during amplification, or direct chemical synthesis of the regulatory clement as well as other methods known in the art. The resulting chimeric regulatory element “variant” can be comprised of the same, or variants of the same, constituent elements of the reference sequence but differ in the sequence or sequences that comprise the linking sequence or sequences which allow the constituent parts to be operatively linked. In the present invention, a polynucleotide sequence provided as SEQ ID NOs:1-8 provide a reference sequence wherein the constituent elements that comprise the reference sequence may be joined by methods known in the art and may comprise substitutions, deletions and/or insertions of one or more nucleotides or mutations that naturally occur in bacterial and plant cell transformation.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e., the introduction of heterologous DNA into a host cell. The term includes an expression cassette isolated from any of the aforementioned molecules.

### Expression Cassettes

The polynucleotide of the present invention can be provided in expression cassettes for expression of a gene of interest in the plant or other organism or host cell of interest. It is recognized that the polynucleotide of the present invention and expression cassettes comprising them can be used for the expression in both human and non-human host cells including, but not limited to, host cells from plants, animals, fungi, and algae. In one embodiment of the invention, the host cells are human host cells or a host cell line that is incapable of differentiating into a human being.

The expression cassette can include 5′ and 3′ regulatory sequences operably linked to the gene of interest to be expressed. “Operably linked” is intended to mean a functional linkage between two or more elements. For example, an operable linkage between one or more genetic regulatory elements and a gene of interest is a functional link between the gene of interest and the one or more genetic regulatory elements that allows for expression of the gene of interest. Operably linked elements may be contiguous or non-contiguous. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes. The expression cassette can include, in the 5′-3′ direction of transcription, a transcriptional and translational initiation region (i.e., a promoter), polynucleotide to be expressed, and a transcriptional and translational termination region (i.e., termination region) functional in plants or other organisms or host cells. The regulatory regions (i.e., promoters, transcriptional regulatory regions, and translational termination regions) and/or the polynucleotide to be expressed may be native/analogous to the host cell or to each other. The promoter may be provided by the polynucleotide of the invention in some embodiments.

Where appropriate, the genes of interest may be optimized for increased expression in the transformed plant. That is, the polynucleotides can be synthesized using plant-preferred codons for improved expression. Methods are available in the art for synthesizing plant-preferred genes. See, for example, U.S. Pat. Nos. 5,380,831, and 5,436,391.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes provided herein may additionally contain 5′ leader sequences. Such leader sequences can act to enhance translation. Translation leaders are known in the art and include: picornavirus leaders, for example, EMCV leader (Encephalomyocarditis 5′ noncoding region); potyvirus leaders, for example, TEV leader (Tobacco Etch Virus), MDMV leader (Maize Dwarf Mosaic Virus), and human immunoglobulin heavy-chain binding protein (BiP); untranslated leader from the coat protein mRNA of alfalfa mosaic virus (AMV RNA 4); tobacco mosaic virus leader (TMV); and maize chlorotic mottle virus leader (MCMV). In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassettes provided herein can also comprise a selectable marker gene for the selection of transformed cells. Selectable marker genes are utilized for the selection of transformed cells or tissues. A selectable marker gene can be positively or negatively selectable. For positive selection, a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of nontransformed plant cells and reducing the possibility of chimeras. Non-limiting exemplary marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, sulfonylurca, glyphosate, glufosinate, L-phosphinothricin, triazine, benzonitrile and 2,4-dichlorophenoxyacetate (2,4-D). Additional selectable markers include phenotypic markers such as β-galactosidase and fluorescent proteins such as green fluorescent protein (GFP), cyan florescent protein (CYP), and yellow florescent protein (YFP). For additional selectable markers, see generally, Yarranton (1992) Curr. Opin. Biotech. 3:506-511; Christopherson et al. (1992) PNAS 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) PNAS 86:5400-5404; Fuerst et al. (1989) PNAS 86:2549-2553; Deuschle et al. (1990) Science 248:480-483; Gossen (1993) Ph.D. Thesis, University of Heidelberg; Reines et al. (1993) PNAS 90:1917-1921; Labow et al. (1990) Mol. Cell. Biol. 10:3343-3356; Zambretti et al. (1992) PNAS 89:3952-3956; Baim et al. (1991) PNAS 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) PNAS 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724; Bourouis et al., EMBO J. 2 (7): 1099-1104 (1983) White et al., Nucl Acids Res 18:1062 (1990), Spencer et al., Theon Appl Genet 79:625-631 (1990), U.S. Pat. Nos. 5,034,322; 6,174,724; 6,255,560; 4,795,855; 5,378,824; and 6,107,549. Such disclosures are herein incorporated by reference.

The above list of selectable marker genes is not limiting. Any selectable marker gene can be used in the present invention. Numerous plant transformation vectors and methods for transforming plants are available. See, for example, An, G. et al. (1986) Plant Pysiol., 81:301-305;Fry, J., et al. (1987) Plant Cell Rep. 6:321-325; Block, M. (1988) Theor. Appl Genet. 76:767-774; Hinchec, et al. (1990) Stadler. Genet. Symp. 203212.203-212; Cousins, et al. (1991) Aust. J. Plant Physiol. 18:481-494; Chec, P. P. and Slightom, J. L. (1992) Gene 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) PNAS 90:11212-11216; Christou, P. (1993) In Vitro Cell. Dev. Biol.-Plant; 29P: 119-124; Davies, et al. (1993) Plant Cell Rep. 12:180-183; Dong, J. A. and Mchughen, A. (1993) Plant Sci. 91:139-148; Franklin, C. I. and Tricu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5:17-27; Eapen et al. (1994) Plant Cell Rep. 13:582-586; Hartman, et al. (1994) Bio-Technology 12:919923; Ritala, et al. (1994) Plant. Mol. Biol. 24:317-325; and Wan, Y. C. and Lemaux, P. G. (1994) Plant Physiol. 104:3748.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. Sec, for example, U.S. Pat. No. 7,250,554, which utilizes P-DNA instead of T-DNA in the transformation vector. Agrobacterium tumefaciens is a naturally occurring bacterium that is capable of inserting its DNA (genetic information) into plants, resulting in a type of injury to the plant known as crown gall. A transgenic plant formed using Agrobacterium transformation methods typically contains a single gene on one chromosome, although multiple copies are possible. Such transgenic plants can be referred to as being hemizygous for the added gene.

Other methods utilized for the delivery foreign DNA or other foreign nucleic acids involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100:247-250; Scheid et al., (1991) Mol. Gen. Genet. 228:104-112; Guerche et al., (1987) Plant Science 52:111-116; Neuhause et al., (1987) Theor. Appl Genet. 75:30-36; Klein et al., (1987) Nature 327:70-73;Howell et al., (1980) Science 208:1265; Horsch et al., (1985) Science 227:1229-1231; DeBlock et al., (1989) Plant Physiology 91:694-701; Methods for Plant Molecular Biology (Weissbach and Weissbach, eds.) Academic Press, Inc. (1988); Methods in Plant Molecular Biology (Schuler and Zielinski, eds.) Academic Press, Inc. (1989); M. E. Fromm et al., Nature, 319, 791 (1986); H. Jones et al., Plant Mol. Biol., 13, 501 (1989) and H. Yang et al., Plant Cell Reports, 7, 421 (1988); UMizuno et al., 2004; Petolino et al., 2000; U.S. Pat. No. 5,302,523; and US Application Publication No. 20040197909; Kaepler et al., 1992; Raloff, 1990; Wang, 1995; U.S. Pat. Nos. 5,204,253; 5,015,580; 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hici et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and Raineri et al., Bio/Tech. 8:33-38 (1990), each of which is incorporated herein by reference in its entirety). The method of transformation depends upon the plant cell to be transformed, stability of vectors used, expression level of gene products and other parameters. Specific methods for transforming certain plant species (e.g., maize, rice, wheat, barley, soybean) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated by reference in its entirety.

Other suitable methods of introducing nucleotide sequences into plant cells and subsequent insertion into the plant genome include microinjection as Crossway et al. (1986) Biotechniques 4:320-334, electroporation as described by Riggs et al. (1986) PNAS 83:5602-5606, Agrobacterium-mediated transformation as described by Townsend et al., U.S. Pat. No. 5,563,055, Zhao et al., U.S. Pat. No. 5,981,840, Yukou et al., WO 94/000977, and Hideaki et al., WO 95/06722, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also sec, Weissinger et al. (1988) Ann. Rev. Genet. 22:421-477; Sanford et al. (1987) Particulate Science and Technology 5:27-37 (onion); Christou et al. (1988) Plant Physiol. 87:671-674(soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) PNAS 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) PNAS 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In some embodiments, the polynucleotides of the invention may be introduced into plants using a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

In some embodiments, genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

In this manner, the present invention provides transformed seed (also referred to as “transgenic seed”) having a polynucleotide construct of the invention, for example, an expression cassette of the invention, stably incorporated into their genome.

In specific embodiments, the nucleic acid molecules and polynucleotide constructs of the present invention can be provided to a plant using a variety of transient transformation methods. Such transient transformation methods include, but are not limited to, the introduction of the sequence or variants and fragments thereof directly into the plant or the introduction of a transcript into the plant. Such methods include, for example, microinjection, electroporation, or particle bombardment. See, for example, Crossway et al. (1986) Mol Gen. Genet. 202:179-185;Nomura et al. (1986) Plant Sci. 44:53-58; Hepler et al. (1994) PNAS 91:2176-2180 and Hush et al. (1994) The Journal of Cell Science 107:775-784, Sheen, J. 2002. A transient expression assay using maize mesophyll protoplasts. Sheen, J. 2001. Signal transduction in maize and Arabidopsis mesophyll protoplasts. Plant Physiol. 2001 December; 127:1466-1475, Anderson et al., U.S. Pat. No. 7,645,919 B2, all of which are herein incorporated by reference. Alternatively, the polynucleotide can be transiently transformed into the plant using techniques known in the art.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefacienscomprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coliorigin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. No. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens(Rogers, et al., Methods in Enzymology 153:253-277 (1987)). Other recombinant vectors useful for plant transformation, including the pCaMVCN transfer control vector, have also been described in the scientific literature (see, for example, Fromm, et al., Proc. Natl. Acad. Sci. USA 82:5824-5828 (1985)).

Various regulatory clements may be included in a construct including any of those provided herein. Any such regulatory elements may be provided in combination with other regulatory elements, e.g., any combination of sequences presented as SEQ ID NO:1-8. Such combinations can be designed or modified to produce desirable regulatory features. In one embodiment, constructs of the present invention comprise at least one regulatory element operably linked to at least one transcribable polynucleotide molecule operably linked to at least one 3′ UTR.

Constructs of the present invention may include any promoter or leader provided herein or known in the art. For example, a promoter of the present invention may be operably linked to a heterologous non-translated 5′ leader such as one derived from a heat shock protein gene (see, for example, U.S. Pat. Nos. 5,659,122 and 5,362,865). Alternatively, a leader of the present invention may be operably linked to a heterologous promoter such as the Cauliflower Mosaic Virus 35S transcript promoter (see, U.S. Pat. No. 5,352,605).

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that affect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Further, when modifying intron/exon boundary sequences, it may be preferable to avoid using the nucleotide sequence AT or the nucleotide A just prior to the 5′ end of the splice site (GT) and the nucleotide G or the nucleotide sequence TG, respectively just after 3′ end of the splice site (AG) to eliminate the potential of unwanted start codons from being formed during processing of the messenger RNA into the final transcript. The sequence around the 5′ or 3′ end splice junction sites of the intron can thus be modified in this manner.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et al., Proc. Natl. Acad. Sci. USA, 80:4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806).

### Transcribable Polynucleotide Molecules

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. The type of DNA molecule can include, but is not limited to, a DNA molecule from the same plant, a DNA molecule from another plant, a DNA molecule from a different organism, or a synthetic DNA molecule, such as a DNA molecule containing an antisense message of a gene, or a DNA molecule encoding an artificial, synthetic, or otherwise modified version of a transgene. Exemplary transcribable DNA molecules for incorporation into constructs of the invention can include, e.g., DNA molecules or genes from a species other than the species into which the DNA molecule is incorporated or genes that originate from, or are present in, the same species, but are incorporated into recipient cells by genetic engineering methods rather than classical breeding techniques.

A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

A promoter of the present invention, such as SEQ ID NOs:1-8, may be operably linked to a transcribable polynucleotide molecule that is heterologous with respect to the promoter molecule. As used herein, the term “heterologous” refers to the combination of two or more polynucleotide molecules when such a combination is not normally found in nature. For example, the two molecules may be derived from different species and/or the two molecules may be derived from different genes, e.g. different genes from the same species or the same genes from different species. Additionally, the two molecules may be derived from isolated locations in the same gene, wherein such a combination of molecules is not normally found in nature. A promoter is thus heterologous with respect to an operably linked transcribable polynucleotide molecule if such a combination is not normally found in nature, i.e., that transcribable polynucleotide molecule is not naturally occurring operably linked in combination with that promoter molecule.

As used herein, the term “overexpression” refers to an increased expression level of a transcribable polynucleotide molecule or a protein in a plant, plant cell or plant tissue, compared to expression in a wild-type plant, cell, or tissue, at any developmental or temporal stage for the gene. Overexpression can take place in plant cells normally lacking expression of a transcribable polynucleotide molecule of interest. Overexpression can also occur in plant cells where endogenous expression of a transcribable polynucleotide molecule or functionally equivalent molecules normally occurs, but such endogenous expression is at a lower level compared to the overexpression. Overexpression thus results in a greater than endogenous production, or “overproduction” of the polypeptide in the plant, cell, or tissue.

In certain embodiments, the expression or overexpression of a transcribable polynucleotide molecule as disclosed herein can affect an enhanced trait or altered phenotype directly or indirectly. In some cases, it may do so, for example, by expressing one or more genes with spatio-temporal precision to effectively increase yield. In certain exemplary embodiments, the protein produced from the transcribable polynucleotide molecule can lead to increased starch content in a plant.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. One of ordinary skill in the art is familiar with using such antisense technology. Briefly, as the antisense transcribable polynucleotide molecule is transcribed, the RNA product hybridizes to and sequesters a complementary RNA molecule inside the cell. This duplex RNA molecule cannot be translated into a protein by the cell's translational machinery and is degraded in the cell. Any gene may be negatively regulated in this manner.

Thus, one embodiment of the invention is a regulatory element of the present invention, such as those provided as SEQ ID NOs:1-8, operably linked to a transcribable polynucleotide molecule so as to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product.

In another embodiment, the transcribable polynucleotide molecule comprises an antisense region of a gene, and the promoter affects the transcription of an antisense RNA molecule, double stranded RNA, or other similar inhibitory RNA molecule in order to inhibit expression of a specific RNA molecule of interest in a target host cell.

### Genes of Agronomic Interest

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, grain composition, product, nutritional profile, disease or pest resistance, environmental or chemical tolerance, and/or may act as a pesticidal agent in the diet of a pest that feeds on the plant. In one embodiment of the invention, a regulatory element of the invention is incorporated into a construct such that the regulatory element is operably linked to a transcribable DNA molecule that is a gene of agronomic interest.

Genes of agronomic interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly.

In a transgenic plant of the present invention, the expression of the gene of agronomic interest can confer a beneficial agronomic trait. A beneficial agronomic trait may include, for example, but is not limited to genes encoding important or desired traits for agronomics, herbicide tolerance or resistance, insect control, insect resistance, modified yield, disease resistance, pathogen resistance, modified plant growth and development, modified starch content, grain characteristics, modified oil content, modified fatty acid content, modified protein content, modified fruit ripening, abiotic stress tolerance, enhanced animal and human nutrition, biopolymer productions, environmental stress resistance, pharmaceutical peptides, sterility, improved processing qualities, improved flavor, hybrid seed production utility, improved fiber production, commercial product production, and biofuel production.

Examples of genes of agronomic interest can include, but are not limited to, genes encoding proteins important for agronomics, such as a yield protein, a stress resistance protein, a developmental control protein, a tissue differentiation protein, a meristem protein, an environmentally responsive protein, a senescence protein, a hormone responsive protein, an insect resistant protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism.

Alternatively, a gene of agronomic interest can affect the above mentioned plant characteristic or phenotype by encoding an RNA molecule that causes the targeted modulation of gene expression of an endogenous gene, for example via antisense (see e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or cosuppression-mediated mechanisms. The RNA could also be a catalytic RNA molecule (e.g., a ribozyme or a riboswitch; see e.g., US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. U.S. Pat. No. 20070124836) and compositions isolated from nematode pests (U.S. Patent Publication No. U.S. Pat. No. 20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species but are incorporated into recipient cells by genetic engineering methods rather than classical reproduction or breeding techniques. The type of polynucleotide molecule can include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

Genes of interest can include, for example, those genes involved in information, such as zinc fingers, those involved in communication, such as kinases, and those involved in housekeeping, such as heat shock proteins; those involved in oil, starch, carbohydrate, or nutrient metabolism; genes encoding enzymes and other proteins from plants and other sources including prokaryotes and other eukaryotes.

Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6, 121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6, 171,640), biopolymers (U.S. Pat. Nos. RE37,543; 6,228,623; and 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Alternatively, a gene of agronomic interest can affect the above mentioned plant characteristics or phenotypes by encoding an RNA molecule that causes the targeted modulation of gene expression of an endogenous gene, for example by antisense (see, e.g., U.S. Pat. No. 5,107,065); inhibitory RNA (“RNAi,” including modulation of gene expression by miRNA-, siRNA-, trans-acting siRNA-, and phased sRNA-mediated mechanisms, e.g., as described in published applications U.S. 2006/0200878 and U.S. 2008/0066206, and in U.S. patent application Ser. No. 11/974,469); or cosuppression-mediated mechanisms. The RNA could also be a catalytic RNA molecule (e.g., a ribozyme or a riboswitch; sec, e.g., U.S. 2006/0200878) engineered to cleave a desired endogenous mRNA product. Methods are known in the art for constructing and introducing constructs into a cell in such a manner that the transcribable DNA molecule is transcribed into a molecule that is capable of causing gene suppression.

In Planta Transient Transformation Methods

The invention is also directed to a method of producing transformed cells and plants which comprise a promoter operably linked to a transcribable polynucleotide molecule.

The term “transformation” refers to the introduction of nucleic acid into a recipient host. As used herein, the term “host” refers to bacteria, fungi, or plant, including any cells, tissue, organs, or progeny of the bacteria, fungi, or plant. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign polynucleotide molecule, such as a construct, has been introduced. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules. The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source or species, is modified from its original form and/or genetic locus; is heterologous to a host cell at least with respect to its location in the genome; the promoter is not the native promoter for the operably linked polynucleotide, and/or is artificially incorporated into a host cell's genome in the current or any prior generation of the cell.

The present invention provides methods of introducing nucleic acids into a recipient host cell or host plant via transient transformation methods. By “transient transformation” is intended that a polynucleotide construct introduced into a plant does not integrate into the genome of the plant. In some embodiments, transient transformation methods are preferred for any plants recalcitrant to stable Agrobacterium transformation, such as legume plants, or specifically cowpea plants. In certain embodiments, methods of in planta transient agrobacterium infiltration (agroinfilatration) are provided. In certain embodiments, an Agrobacterium tumefaciens strain such as AGL1, C58C1, EHA105, or GV3101 is transformed with a genetic construct as described herein, preferably AGLI. Recipient plants may be grown to various vegetative stages before infiltration, preferably early vegetative stages. Recipient cowpea plants may be grown, for example, to about 2 weeks to about 5 weeks before infiltration. Recipient Nicotiana benthamiana plants may be grown, for example, to about 4 weeks to about 6 weeks before infiltration.

Agrobacterium inoculum for infiltration may be at any concentration, preferably between an OD600 of about 0.1 to about 0.8, most preferably between an OD600 of about 0.3 to about 0.5. In certain embodiments, infiltration of the youngest fully expanded trifoliate leaves are preferred, such as fully expanded trifoliate leaves between about 2 weeks to about 5 weeks, preferably about 2 weeks.

In certain examples, gene expression in transiently transformed leaves may be evaluated in leaf punches, for example using a detectable marker.

### Stable Transformation Methods

DNA molecules of the present invention may also be introduced into host cells or host plants using stable transformation methods known in the art. “Stable transformation” is intended that the polynucleotide construct introduced into a plant integrates into the genome of the plant and is capable of being inherited by progeny thereof. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol. 42:205 (1991)), gene editing (e.g., CRISPR-Cas systems), among others.

Technology for stable introduction of a DNA molecule into cells is well known to those of skill in the art. Methods and materials for transforming plant cells by introducing a plant DNA construct into a plant genome in the practice of this invention can include any of the well-known and demonstrated methods. Any transformation methods may be utilized to transform a host cell with one or more promoters and/or constructs of the present. Host cells may be any cell or organism such as a plant cell, algae cell, algae, fungal cell, fungi, bacterial cell, or insect cell. Preferred hosts and transformed cells include cells from: plants, Aspergillus, yeasts, insects, bacteria, and algae. In specific embodiments, the host cells and transformed cells may include cells from crop plants.

In various embodiments, the methods described herein can involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

A transgenic plant subsequently may be regenerated from a transgenic plant cell of the invention. Using conventional breeding techniques or self-pollination, seed may be produced from this transgenic plant. Such seed, and the resulting progeny plant grown from such seed, will contain the recombinant DNA molecule of the invention, and therefore will be transgenic.

Regenerated transgenic plants can be self-pollinated to provide homozygous transgenic plants (homozygous for the recombinant DNA molecule). Alternatively, pollen obtained from the regenerated transgenic plants may be crossed with non-transgenic plants to provide seed for heterozygous transgenic plants (heterozygous for the recombinant DNA molecule), preferably inbred lines of agronomically important species. Both such homozygous and heterozygous transgenic plants are referred to herein as “progeny plants.” Progeny plants are transgenic plants descended from the original transgenic plant and containing the recombinant DNA molecule of the invention. Seeds produced using a transgenic plant of the invention can be harvested and used to grow generations of transgenic plants, i.e., progeny plants of the invention, comprising the construct of this invention and expressing a gene of agronomic interest. Descriptions of breeding methods that are commonly used for different traits and crops can be found in one of several reference books, see, for example, Allard, Principles of Plant Breeding, John Wiley & Sons, NY, U. of CA, Davis, CA, 50-98 (1960); Simmonds, Principles of crop improvement, Longman, Inc., NY, 369-399 (1979); Sneep and Hendriksen, Plant breeding perspectives, Wageningen (ed), Center for Agricultural Publishing and Documentation (1979); Fehr, Soybeans: Improvement, Production and Uses, 2nd Edition, Monograph, 16:249 (1987); Fehr, Principles of variety development, Theory and Technique, (Vol. 1) and Crop Species Soybean (Vol 2), Iowa State Univ., Macmillan Pub. Co., NY, 360-376 (1987). Conversely, pollen from non-transgenic plants may be used to pollinate the regenerated transgenic plants.

The transformed plants may be analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. The expression of a transcribable polynucleotide molecule can be measured using TaqMan® (Applied Biosystems, Foster City, CA) reagents and methods as described by the manufacturer and PCR cycle times determined using the TaqMan® Testing Matrix. Alternatively, the Invader® (Third Wave Technologies, Madison, WI) reagents and methods as described by the manufacturer can be used to evaluate transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. Plant parts of the invention may be viable, nonviable, regenerable, and/or non-regenerable. The invention also includes and provides transformed plant cells comprising a DNA molecule of the invention. The transformed or transgenic plant cells of the invention include regenerable and/or non-regenerable plant cells. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immuno-precipitation, and ELISA.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof. A commodity product of the invention will contain a detectable amount of DNA corresponding to the recombinant DNA molecule of the invention. Detection of one or more of this DNA in a sample may be used for determining the content or the source of the commodity product. Any standard method of detection for DNA molecules may be used, including methods of detection disclosed herein.

### Plants

The nucleic acid molecules and polynucleotide constructs of the present invention can be used for transformation of any plant species, including, but not limited to, monocots and dicots. Examples of plant species of interest include, but are not limited to, Arabidopsis thaliana , peppers (Capsicum spp; e.g., Capsicum annuum, C. baccatum, C. chinense, C. frutescens, C. pubescens, and the like), cowpea (Vigna unguiculata), tomatoes (Lycopersicon esculentum), tobacco (Nicotiana tabacum), eggplant (Solanum melongena), petunia (Petunia spp., e.g., Petunia x hybrida or Petunia hybrida), corn or maize (Zea mays), Brassica ssp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), green millet (Setaria viridis), finger millet (Eleusine coracana)), sunflower (Helianthus annuus), safflower (Carthamus tinctorius), wheat (Triticum aestivum), soybean (Glycine max), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), switchgrass (Panicum virgatum), algac (e.g., Chlamydomonas reinhardtii, Botryococcus braunii, Chlorella spp., Dunaliella tertiolecta, Gracilaria spp.), oats, barley, vegetables, ornamentals, and conifers. The nucleic acid molecules and polynucleotide constructs of the present invention can also be used for transformation of any algae species.

As used herein, the term “plant” refers to any living organism belonging to the kingdom Plantae (i.e., any genus/species in the Plant Kingdom). In some embodiments, the plant is a tree, herb, bush, grass, vine, fern, moss, or green algae. The plant may be monocotyledonous (monocot), or dicotyledonous (dicot). Examples of particular plants that may comprise a polynucleotide of the invention include but are not limited to Arabidopsis, Brachypodium, switchgrass, corn, potato, rose, apple tree, sunflower, wheat, rice, bananas, plantains, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, cauliflower, cavalo, collards, cowpea, kale, kohlrabi, mustard greens, rape greens, and other brassica leafy vegetable crops, bulb vegetables (e.g., garlic, leck, onion (dry bulb, green, and Welch), shallot), citrus fruits (e.g., grapefruit, lemon, lime, orange, tangerine, citrus hybrids, pummelo), cucurbit vegetables (e.g., cucumber, citron melon, edible gourds, gherkin, muskmelons (including hybrids and/or cultivars of Cucumis melons), water-melon, cantaloupe, and other cucurbit vegetable crops), fruiting vegetables (including eggplant, ground cherry, pepino, pepper, tomato, tomatillo), grape, leafy vegetables (e.g., romaine), root/tuber and corm vegetables (e.g., potato, yam, cassava, taro), tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, blackberries, cloudberries, loganberries, raspberries, salmonberries, thimbleberries, and wineberries), cereal crops (e.g., corn (maize), rice, wheat, barley, sorghum, millets, oats, ryes, triticales, buckwheats, fonio, quinoa, oil palm), Brassicaceae family plants, and Fabaceae family plants, pome fruit (e.g., apples, pears), stone fruits (c.g., coffees, jujubes, mangos, olives, coconuts, oil palms, pistachios, almonds, apricots, cherries, damsons, nectarines, peaches and plums), vine (e.g., table grapes, wine grapes), fiber crops (e.g., hemp, cotton), ornamentals, and the like.

Having now generally described the invention, the same will be more readily understood through reference to the following examples which are provided by way of illustration and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: Development of an In Planta Transient System for Genetic Manipulation of Cowpea

Like other grain legume crops, cowpea (Vigna unguiculata L. Walp) is recalcitrant to Agrobacterium-mediated transformation. This has previously limited genetic manipulation to produce cowpea lines with enhanced agronomic properties such as tolerance to abiotic and biotic stressors.

In order to allow for efficient genetic manipulation of cowpea, the present inventors have developed an in planta transient system based on Agrobacterium infiltration using attached trifoliate leaves of cowpea. Effective agrobacterium strains and inoculum concentrations for cowpea have been determined. This novel system demonstrates efficient expression of a reporter gene in the youngest fully expanded leaf across different growth stages. This achievement has enabled determination of in planta expression levels of promoters identified from RNA sequencing (RNA-seq), which constitutively express with different strengths. Furthermore, this technology allows for subjecting whole plants to drought stress in order to identify drought-inducible promoters. These findings provide a platform for validation of regulatory elements and genes within intact plants, particularly pertinent for legume species that were previously limited by genetic differences. The present disclosure further expands the genetic resources available to researchers by assembling RNA-seq data from different canopy levels and developmental stages in cowpca.

### Example 2: Agro-transformation of Cowpea

AGL1 strain is effective for transient expression in intact cowpea leaves

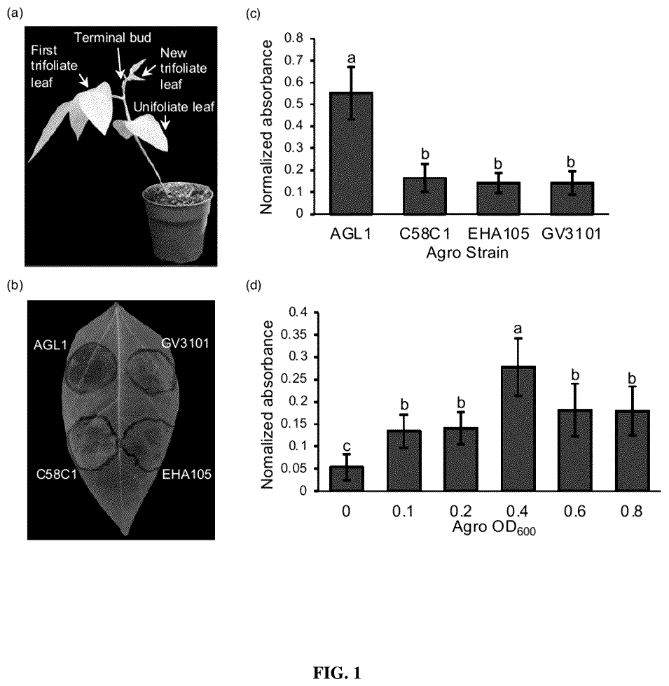

A reporter construct (pRUBY) expressing a polycistronic mRNA containing three betalain biosynthetic genes driven by the Cauliflower Mosaic Virus (CaMV) 35S promoter was used to determine the most effective strain of agrobacterium for transforming cowpea. Betalain accumulation produces red pigmentation that is visible by eye and can be quantified via absorbance measurements at 535 nm. The agrobacterium strains AGL1, C58C1, EHA105, and GV3101 were transformed with the pRUBY construct and infiltrated into the first trifoliate leaf from 2-week-old cowpea plants (FIG. 1, panel (a)). All agrobacterium strains resulted in betalain accumulation in the leaf without senescence or cell death (FIG. 1, panel (b)), however the AGLI-infiltrated leaf showed consistently higher betalain accumulation compared to the other agrobacterium strains (FIG. 1, panel (c)). The efficiency of the transient assay was further improved by testing different inoculum concentrations using the AGLI strain. Among cell densities ranging from 0.1 to 0.8 at OD₆₀₀, an OD₆₀₀ of 0.4 produced the highest betalain accumulation (FIG. 1, panel (d)). Therefore, AGLI at an OD₆₀₀ of 0.4 was used for further experiments.

### Youngest Fully Expanded Leaf is Highly Amenable to Agro-Transformation

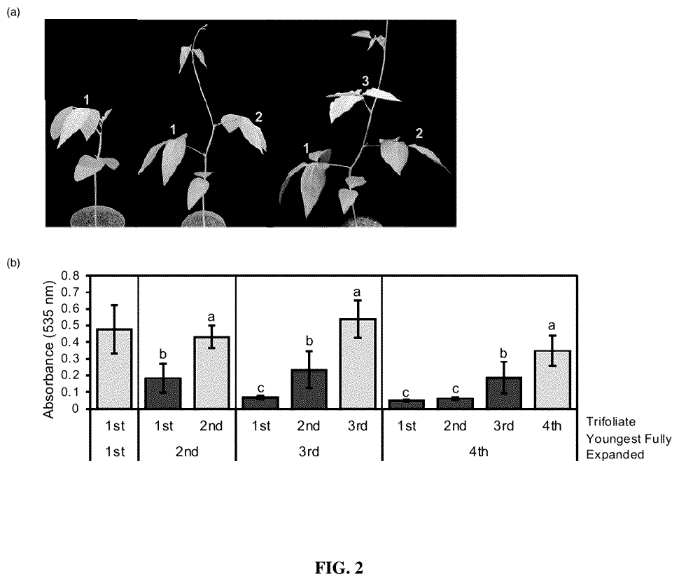

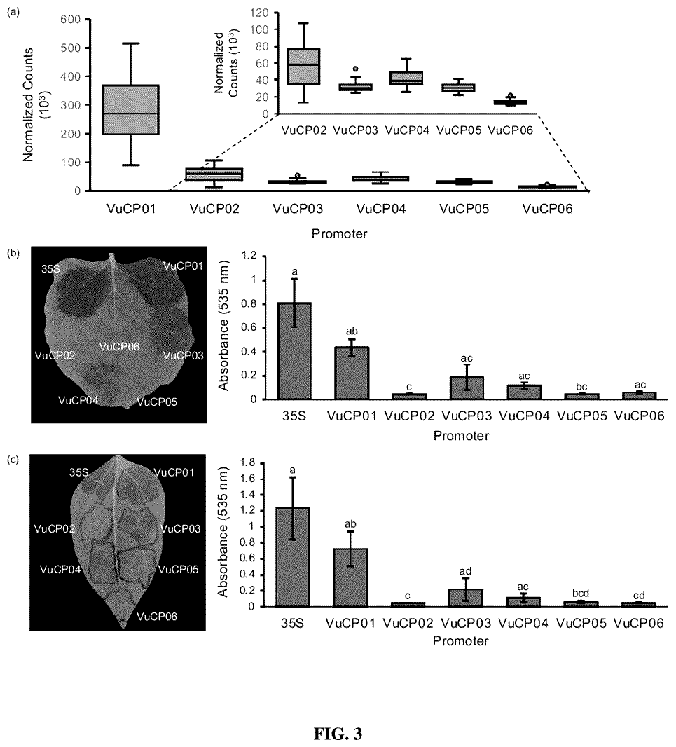

Initial experiments revealed leaf-to-leaf variability in transient gene expression, and a possible developmental component to Agrobacterium susceptibility in cowpea was further investigated. Cowpea plants were grown to various early vegetative stages, from 2 to 5 weeks, and fully expanded trifoliate leaves were infiltrated (FIG. 2, panel (a)). It was observed that the youngest fully expanded trifoliate showed the highest betalain accumulation at all stages of plant development (FIG. 2, panel b). Although most of the leaves retained some capacity for transient gene expression, these results indicate that the youngest fully expanded leaf is a highly effective developmental stage for use in transient gene expression assays.

### Example 3: Experimental Procedures for Agro-transformation of Cowpea

### Plant Materials and Growth Conditions

Cowpea (Vigna unguiculata L. Walp) line IT97K-499-35 (provided by University of California, Riverside) was used for all experiments. Plants used for optimizing transient expression were grown in a Conviron growth chamber under a 12 h/12 h light/dark photoperiod at 25/23° C. and 65% relative humidity. During light periods, the PPFD was approximately 400 μmol m⁻² s⁻¹. Two seeds were sown 6 cm apart per pot with Berger BM7 Bark Mix (Quebec, Canada). Plants used for RNA-Seq were planted in-ground (3 inches between plants and 30 inches between rows) and maintained in a greenhouse with irrigation, weekly fertilizer application, and supplemental lighting (14 h/10 h photoperiod with 1000 μmol/m²·s supplemental light). Nicotiana benthamiana were grown at a PPFD of 120 μmol m⁻² s⁻¹ for 5 weeks in a Conviron growth chamber under a 12 h/12 h light/dark photoperiod at 25/22° C. and 65% relative humidity.

### Agrobacterium Infiltration

The binary vector, pRUBY (Addgene; 160908), was transformed into Agrobacterium tumefaciens strains AGL1, C58C1, EHA105, and GV3101 using either chemical or electroporation-based methods. The Agrobacterium cells used in the experiments were inoculated into 2 mL of Luria broth (LB) media in addition to appropriate antibiotics for 48 hours at 28° C., and then 20 μL was refreshed into 5 mL of LB media for 24 hours at 28° C. with shaking at 200 rpm. Cultures were centrifuged at 4000 rpm for 5 minutes and resuspended in infiltration medium consisting of 10 mM of MES monohydrate, pH 5.6 with KOH, 10 mM of MgCl₂, 150 μM Acetosyringone, 0.5% (w/v) of glucose and 0.25% (w/v) of MS salts. The agrobacterium was incubated for 1 hour under dark conditions before infiltration with an OD₆₀₀ of 0.4. The central leaf of the first trifoliate was selected for agroinfiltration. Cowpea plants were around 2 weeks old, and the first trifoliate leaves were fully expanded when used in the experiment. Prior to the infiltration, small wounds were created in the upper epidermis with a needle. A blunt 1 ml syringe was then used to infiltrate the adaxial part of the cowpea leaves until a waterlogged region of adequate area was formed within the leaf tissue. When Nicotiana benthamiana plants were approximately 5 weeks in age, they were infiltrated with GV3101 agrobacterium at an OD600 of 0.4 following the same methodology described in Leonelli, (2022).

### Betalain Quantification

3 days following infiltration, 7/16″ diameter leaf punches were taken from the inoculated regions and placed into 2ml screw-cap collection tubes with lysing matrix D resin beads (MP Biomedicals, cat #: 116540434). The tubes were frozen at −80° C. and the samples were shaken vigorously using a TissueLyser II to disrupt the tissue, then 200 μL of 10 mM MES pH 5.6 was added to each sample. The samples were mixed by vortexing then centrifuged at maximum speed for 3 minutes. The supernatant was then syringe filtered through 0.45 um nylon filters to remove any residual plant tissue. 100 μL of supernatant from each sample was pipetted into the wells of a 96-well plate and absorbance measurements were taken at 535 nm using a Tecan plate reader.

### RNA-Seq

Five replicates (3×½″ diameter leaf punches) were collected from the top, middle, and bottom canopy positions of cowpea at growth stages R1 (˜5.5 weeks after planting) and R3 (˜6.5 weeks after planting). RNA was purified from these 30 samples using the MagMAX kit for RNA extraction (ThermoFisher Scientific, cat #AM1830) and libraries were made with the Kapa HyperPrep mRNA kit (Roche) library kit according to the manufacturer's protocol. Libraries were barcoded with Unique Dual Indexes (UDI's) which have been developed to prevent index switching. Final libraries were quantified with Qubit (ThermoFisher) and the average cDNA fragment sizes were determined on a Fragment Analyzer. The libraries were diluted to 10 nM and further quantitated by qPCR on a CFX Connect Real-Time qPCR system (Biorad) for accurate pooling of barcoded libraries and maximization of number of clusters in the flowcell. Samples were sequenced using a Novaseq SP lane to generate 150 nucleotide paired-end reads with roughly 20 million reads per sample.

### Promoter Selection

Raw reads were aligned to the cowpea reference genome via HISAT2 and read counts were generated with the featureCounts program (Kim et al., 2019; Liao, et al., 2014). Differential expression analysis was carried out in the statistical programming language R using RStudio software with several add-on packages including DESeq2 (Love, 2014) and Shiny (Chang, 2023). The DESeq2 package was used for normalizing raw RNA-Seq read counts and performing differential expression analysis, and the shiny package was used to construct an interactive application to filter for the constitutively expressed genes whose promoters identified and described herein. Genes were filtered by normalized counts to look for constitutive highly expressed gene candidates expressed across all sampled time points and canopy positions. Gene candidate filtering was performed using an interactive R Shiny application developed by the inventors which allows users to filter for genes which meet adjusted p-value and overall expression cutoff values, and which have a specified canopy expression profile. Drought-responsive gene candidates were selected. Potential promoter regions corresponding to each gene candidate ranging from around 1700-2500 bp in length and located immediately upstream of the start codon were selected. The genomic binding regions of the primers were designed such that the reverse primer binds directly upstream of the start codon, and the forward primer was selected using Primer Blast (NCBI) to ensure specific binding.

### Vector Construction

A modified pRUBY expression vector was cloned using a Golden Gate cloning workflow to add a GFP expression cassette (GFPdo) upstream of the RUBY reporter, creating pRUBY-GFPdo. To clone the promoter candidates into this modified vector, the GFPdo cassette was first removed with BsaI-HFv2 (NEB), and the vector backbone was purified. The promoter candidates were then PCR amplified from purified cowpea genomic DNA (NEB Q5 DNA Polymerase; Qiagen DNeasy Plant Pro Kit), with primers designed to add BsaI or BsmBI (NEB) restriction sites and complementary overhangs. Six constitutive and two drought-responsive promoter candidates were amplified, digested, and ligated (NEB Quick Ligase) into the digested pRUBY-GFPdo backbone to drive expression of the RUBY reporter cassette. Sequence verified vectors were then transformed as described above into AGLI for cowpea transient assays, or GV3101 for Nicotiana benthamiana transient assays.

### Drought Treatment and Physiological Analysis

Plants were exposed to drought stress a day after transient expression assay. Fast drought stress was induced by gently removing the soil and wrapping the roots in tissue paper. Slow drought stress was induced by interrupting water supply. Stomatal conductance and quantum yield of photosystem II (ΦPSII) were measured using a LI-600 porometer/fluorometer (LI-COR Bioscience) to determine the level of drought stress. The measurements were taken in the empty areas of the infiltrated leaves.

### Statistics

Raw absorbance data in FIG. I was normalized by dividing each absorbance value by the sum of absorbances from that replicate to correct the variation between leaves. Some replicates showed very little to no betalain expression and were not included in the analysis. The percentage of replicates which showed expression and were included in the analysis was calculated for each experiment. After the method was established, raw absorbance data of betalain was represented in FIG. 2-4.

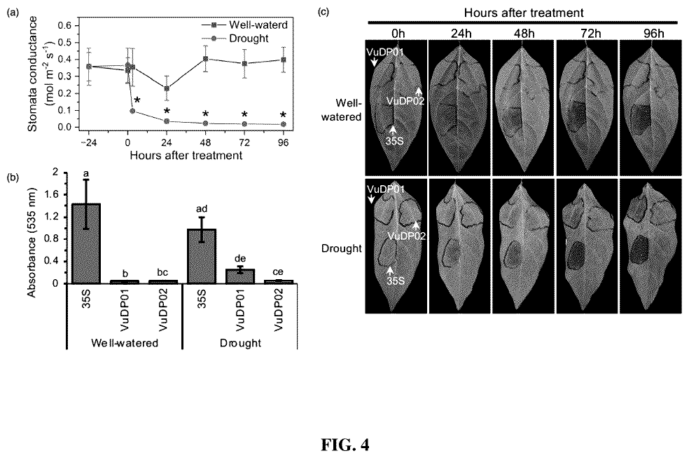

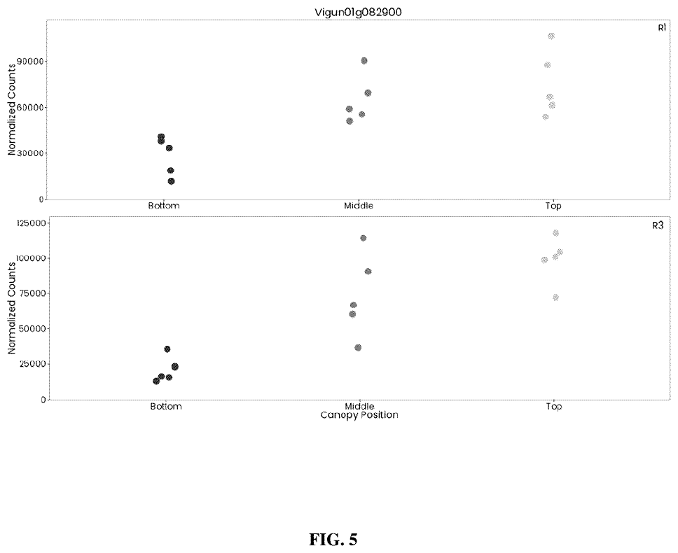

Influential outliers were determined with Cook's distances using a cutoff of 4/n and were not included in the analysis. Prior to analyzing statistical significance, the data was checked for constant variance and normality using the Breusch-Pagan and Shapiro-Wilk tests, respectively. If these tests passed with a significance level of 0.05, one-way ANOVA was performed to test for multiple groups with equal means, followed by pairwise t-tests (Bonferroni correction, confidence level of 95%). If the equal variance and normality assumptions did not hold, a Kruskal-Wallis test was used followed by Dunn's test with the Benjamini-Hochberg correction. Mann-Whitney test was used to compare well-watered control group and drought stress group of the stomatal conductance and photosystem II efficiency in FIG. 4.

### Example 4: Evaluation of Cowpea Promoters Using Transient Expression Assay

Using the newly developed optimized cowpea agroinfiltration method, several candidate promoters from cowpea were tested to identify new constitutive and global promoters for genetic engineering efforts in cowpea. Candidate promoters were selected using RNA-seq data filtered for genes whose expression remained similar in samples collected from cowpea at R1 and R3 developmental stages and at top, middle, and bottom canopy positions. In order to facilitate the selection of candidate promoters, an application called CowPEAsy (https://jake-harris.shinyapps.io/cowpeasy_beta/) was developed using the Shiny package in R to filter and visualize the cowpea RNA-seq expression data provided herein for constitutively expressed genes. Six candidate promoters were selected by filtering for genes whose expression remained similar in different developmental stages and at all sampled canopy positions with varied strengths (FIG. 3, panel (a); Table 1). An application was developed using the shiny package in R to facilitate the filtering and visualization of cowpea RNA-seq expression data for constitutively expressed genes (FIG. 5). For each gene of interest, promoter regions were selected ranging from between roughly 1700 bp to 2500 bp in length and ending immediately upstream of the start codon. This range of promoter candidate lengths was necessary to avoid extensive non-specific primer binding. Six candidate promoters (VuCP01 to VuCP06; SEQ ID NOs: 1-6; Table 1) showed a range of expression strengths in both Nicotiana benthamiana and in cowpea when used to transiently drive expression of a RUBY reporter in a modified pRUBY vector (pRUBY-GFPdo) containing a GFP dropout cassette directly upstream of the RUBY reporter (FIG. 3, panels (a)-(c)). Transient expression of these six constitutive promoter expression cassettes was carried out as described herein. At 72 hours post-infiltration, a range of betalain accumulation levels were observed, with one highly expressed promoter (VuCP01) showing approximately 60% of the 35S promoter control. Three other promoters (VuCP03, VuCP04, VuCP05) showed moderate to low expression.

Promoters VuCP05 and VuCP06 did not show a statistically significant difference in expression. However, qualitatively, as seen in the leaf pictures of FIG. 3, VuCP05 showed a low level of expression when tested in cowpea, and VuCP06 showed a low level of expression when tested in N. benthamiana. Both VuCP05 and VuCP06 showed very little to no expression across both species tested (FIG. 3, panel (b)). Despite the high level of expression seen in the gene candidate corresponding to VuCP02 (FIG. 3, panels (b) and (c)), no expression was seen with this promoter candidate in either cowpea or N. benthamiana.

TABLE 1
Cowpea promoter candidate locations and normalized RNA-Seq read counts.

| | SEQ | | | | | | | |
|---|---|---|---|---|---|---|---|---|
| | ID | | R1 Mean Read Counts | | | R3 Mean Read Counts | | |
| Promoter | NO | GeneID | Bottom | Middle | Top | Bottom | Middle | Top |
| VuCP01 | 1 | Vigun01g219300 | 169078.9 | 321567.8 | 252557.5 | 221665.6 | 393395.0 | 400517.4 |
| VuCP02 | 2 | Vigun11g163500 | 68636.8 | 66721.7 | 49142.7 | 63840.7 | 49188.3 | 49938.8 |
| VuCP03 | 3 | Vigun04g096500 | 31138.9 | 29716.0 | 30853.3 | 31570.2 | 31771.6 | 34868.0 |
| VuCP04 | 4 | Vigun08g150700 | 38900.8 | 43994.1 | 32967.2 | 36149.3 | 48968.9 | 51555.3 |
| VuCP05 | 5 | Vigun09g108850 | 30710.6 | 31572.5 | 26426.0 | 35378.7 | 32436.6 | 26530.0 |
| VuCP06 | 6 | Vigun02g039700 | 15012.0 | 15936.3 | 13207.0 | 10780.4 | 12441.8 | 13955.1 |

### Example 5: Transiently Expressed Drought-Inducible Promoters Respond to Whole-Plant Stress in Cowpea

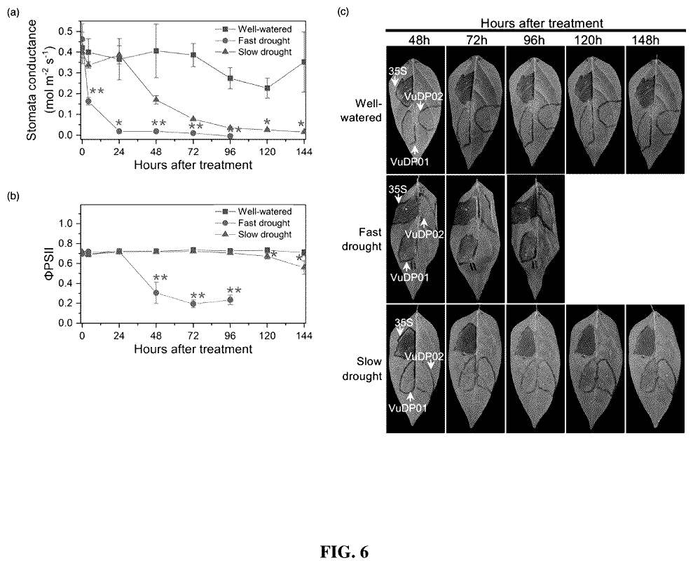

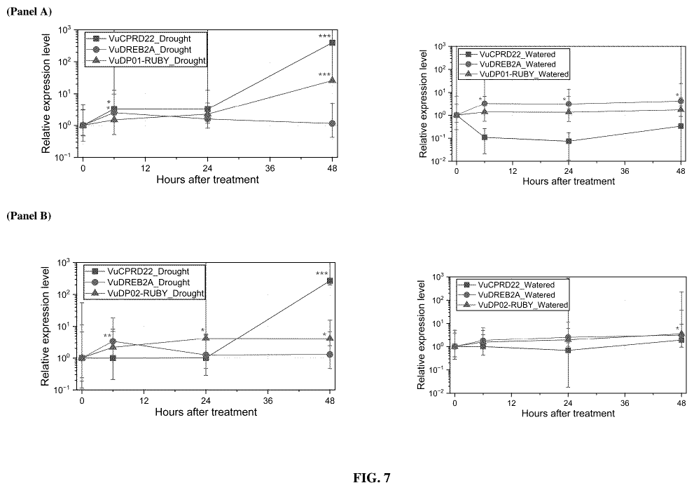

One advantage of developing a transient gene expression assay in intact plants is the ability to test how genes or regulatory elements respond to various treatments in physiologically relevant contexts. The novel transient gene expression system described herein was used to characterize candidate promoters VuDP01 (SEQ ID NO:7) from the VuCPRD22 gene and VuDP02 (SEQ ID NO:8) from the VuDREB2A gene, previously investigated (Iuchi, et al., 1996; Sadhukhan, et al., 2014). Sequences upstream of the ATG start codon of these genes were selected and cloned into the promoter region of the pRUBY vector using a Golden Gate cloning strategy. After transient expression of the vectors containing the candidate promoters in cowpea, the infiltrated plants were exposed to drought stress for 96 or 144 hours and monitored for stomatal conductance, quantum yield of photosystem II, and betalain accumulation (FIG. 4 and FIG. 6). Plants showed a decrease in stomatal conductance after 3 hours and by 24 hours the stomata were completely closed to counteract dehydration (0.036 mol m⁻2 s⁻¹; FIG. 4, panel (a)). Drought stress was further evidenced by a reduction in photosystem II efficiency after 48 hours as measured by reduced gas exchange resulting from stomatal closure (FIG. 6, panel (b)).

By 96 hours post inoculation, betalain accumulation in leaf spots infiltrated with the VuDP01 promoter construct reached 26% of the 35S positive control betalain levels, whereas in well-watered control plants, the VuDP01 promoter construct never accumulated betalain (FIG. 4, panels (b) and (c)). Using CowPEAsy, it was determined that VuCPRD22 and VuDREB2A showed low expression levels in the RNAseq data collected from well-watered plants (Table 2). The induction of RUBY expression driven by the 2 kb region upstream of the VuCPRD22 gene indicates that this region acts as a drought-inducible promoter. Interestingly, the VuDP02 promoter did not show betalain accumulation in either drought or well-watered conditions (FIG. 4, panels (b) and (c)). This suggests that cis-regulatory elements in the 2 kb upstream of VuDREB2A gene are not alone sufficient to drive gene expression in response to drought stress. In addition to the 1.9 kb promoter region suggested by in silico analysis (Sadhukhan et al., 2014), there might be regulatory elements required in more distal regions to induce expression of VuDREB2A. In plants subjected to slow drought stress (by interrupting water supply), significant differences in stomatal conductance and photosystem II efficiency began showing at 120 hours after water was withheld and betalain accumulation driven by the VuDPO1 promoter was slower than in the plants subjected to the fast drought stress (FIG. 6).

TABLE 2
Normalized RNA-Seq read counts of drought inducible genes,
VuDP01 and VuDP02.

| | | R1 Mean Read Counts | | | R3 Mean Read Counts | | |
|---|---|---|---|---|---|---|---|
| Promoter | GeneID | Bottom | Middle | Top | Bottom | Middle | Top |
| VuDP01 | Vigun04g020700 | 25.4 | 7.9 | 1.5 | 6.0 | 2.1 | 7.5 |
| VuDP02 | Vigun08g165400 | 93.8 | 84.7 | 56.1 | 64.2 | 52.7 | 41.8 |

### Example 6: Further Evaluation of Drought-Inducible Promoters

Expression levels of qPCR reference genes (VuRPL40e and VuUE21D), drought-responsive native genes (VuDREB2A and VuCPRD22), and RUBY under the control of drought-inducible promoters (VuDP01, SEQ ID NO:7, and VuDP02, SEQ ID NO:8) was evaluated using qPCR. qPCR showed that the native genes (VuDREB2A and VuCPRD22) respond to drought, and that the drought-inducible promoters disclosed herein, particularly VuDP01, also respond to drought, leading to the transcript accumulation (FIG. 7, Panels A and B).

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

1. A DNA molecule comprising a DNA sequence selected from the group consisting of:
a) a sequence with at least 85 percent sequence identity to any of SEQ ID NOs:2 and 4-8;
b) a sequence comprising any of SEQ ID NOs:2 and 4-8;
c) a fragment of a sequence having at least 85 percent sequence identity to any of SEQ ID NOs:2 and 4-8, wherein the fragment has gene-regulatory activity;
d) a fragment of any of SEQ ID NOs:2 and 4-8, wherein the fragment has gene-regulatory activity; and
e) combinations thereof,
wherein said sequence is operably linked to a heterologous transcribable polynucleotide molecule.

2. The DNA molecule of claim 1, wherein;
a) the DNA sequence is active as a promoter;
b) the DNA molecule further comprises a heterologous regulatory element; and/or
c) the DNA sequence comprises gene regulatory activity.

3. (canceled)

4. The DNA molecule of claim 1, wherein said sequence has at least 90 percent sequence identity to the DNA sequence of any of SEQ ID NOs: 2 and 4-8.

5. The DNA molecule of claim 1, wherein said sequence has at least 95 percent sequence identity to the DNA sequence of any of SEQ ID NOs: 2 and 4-8.

6. (canceled)

7. The DNA molecule of claim 1, wherein the heterologous transcribable polynucleotide molecule comprises a gene of agronomic interest.

8. The DNA molecule of claim 7, wherein:
a) the gene of agronomic interest confers increased yield, increased root growth, or increased drought resistance in plants; and/or
b) the gene of agronomic interest confers increased starch content in plants.

9. (canceled)

10. A construct comprising at least one copy of a DNA molecule of claim 1, and an operably linked transcribable gene of agronomic interest.

11. The construct of claim 10, wherein the construct comprises in the 5′-3′ direction:
(a) the at least one copy of said DNA molecule; (b) the operably linked transcribable gene of agronomic interest; and (c) a gene termination sequence.

12. The construct of claim 10, wherein the transcribable gene of agronomic interest comprises an open reading frame encoding a polypeptide.

13. A transgenic plant cell comprising a heterologous DNA molecule comprising a sequence selected from the group consisting of:
a) a sequence with at least 85 percent sequence identity to any of SEQ ID NOs:2 and 4-8;
b) a sequence comprising any of SEQ ID NOs:2 and 4-8;
c) a fragment of a sequence having at least 85 percent sequence identity to any of SEQ ID NOs:2 and 4-8, wherein the fragment has gene-regulatory activity;
d) a fragment of any of SEQ ID NOs:2 and 4-8, wherein the fragment has gene-regulatory activity; and
e) combinations thereof,
wherein said sequence is operably linked to a heterologous transcribable polynucleotide molecule.

14. The transgenic plant cell of claim 13, wherein said transgenic plant cell is a monocotyledonous plant cell or a dicotyledonous plant cell.

15. (canceled)

16. A transgenic plant, transgenic plant part, transgenic progeny plant, or transgenic seeder part thereof, comprising the DNA molecule of claim 1.

17-18. (canceled)

19. A method of producing a commodity product comprising obtaining a transgenic plant or part thereof according to claim 16 and producing the commodity product therefrom.

20. The method of claim 19, wherein the commodity product is selected from the group consisting of protein concentrate, protein isolate, grain, starch, seeds, meal, flour, biomass, or and seed oil.

21. A commodity product comprising the DNA molecule of claim 1.

22. The commodity product of claim 21, wherein the commodity product is selected from the group consisting of protein concentrate, protein isolate, grain, starch, seeds, meal, flour, biomass, er and seed oil.

23. A method of expressing a transcribable polynucleotide molecule comprising obtaining a transgenic plant according to claim 16 and cultivating the plant, wherein the transcribable polynucleotide is expressed.

24. A method of expressing a gene of agronomic interest in a plant or plant cell, the method comprising incorporating into a plant cell a construct comprising the DNA molecule of claim 1, operably linked to a the gene of agronomic interest, wherein the DNA molecule is capable of driving the expression of the operably linked gene of agronomic interest in the plant cell.

25. The method of claim 24, further comprising regenerating a transformed plant from said plant cell.

26. The method of claim 24, wherein said plant cell is stably transformed with said construct.

27. A method of producing a transgenic plant cell comprising introducing the DNA molecule of claim 1 into a plant cell.

28. The method of claim 27, wherein introducing said DNA molecule into said plant cell comprises transformation.

29. The method of claim 28, further comprising regenerating a transgenic plant from said plant cell.

30. The method of claim 27, wherein introducing said DNA molecule into said plant cell comprises crossing the transgenic plant of claim 16 with another plant to produce a progeny plant comprising said plant cell.

31. A method of identifying a regulatory element or expressing a gene in an attached trifoliate leaf of a plant comprising:
incorporating into the attached trifoliate leaves of the plant an effective amount of an agrobacterium strain producing a betalain concentration greater than the betalain concentrations of agrobacterium strains C58C1, EHA105, or GV3101.

32. The method of claim 31, wherein
a) the effective amount is a cell density between about 0.1 and about 0.8, between about 0.2 and about 0.6, or about 0.4 at OD₆₀₀;
b) the agrobacterium strain producing a betalain concentration greater than the betalain concentrations of agrobacterium strains C58C1, EHA105, or GV3101 is AGL1;
c) the incorporating of the agrobacterium strain does not require senescence or cell death
d) the attached trifoliate leaf of the plant is the youngest fully expanded trifoliate; and/or
e) the agroinfiltrating comprises incorporating into a plant cell a construct comprising the DNA molecule of claim 1.

33-36. (canceled)

37. The method of claim 31, further comprising identifying genes or constitutive or global promoters or regulatory elements for the plant.

38. The method of claim 31, further comprising subjecting the plant to physiological stress conditions.

39. The method of claim 38, wherein the physiological stress condition comprises:
a) an unfavorable condition that affects a plant's metabolism, growth, or development;
b) _an abiotic stress or environmental factors (e.g., extreme cold or hot temperature, excessive watering, drought, salinity of the soil); of
c) a biotic stress or biological factors (e.g., disease causing microorganisms (e.g., bacteria, fungi, and virus), pests, or insects; and/or
d) temperature stress; stress due to a lack or excess of light; stress produced by infections from pathogens, pests, or viruses; stress due to absence or excess of nutrients; salinity stress; water stress; or mechanical stress.

40. (canceled)

41. The method of claim 38, further comprising identifying stress-inducible genes or regulatory elements.

42. The method of claim 31, wherein the regulatory element is identified by measuring betalain accumulation in a modified plant compared to a control plant.


SEQUENCE LISTING (8 sequences)

SEQ ID NO: 1 - DNA, 1697 bp, Vigna unguiculata
  source 1..1697: mol_type = genomic DNA
agtgcagatagaataaacagccactttacaattgtattttaatgatataataatatatatgtatattttaatttaagaaataaaaggtatgtattaaagtattagcatgataagttattgtcgtttatttattttgagaagtcaattatcaatactcttatttaagaatgcaatttgtttgacgaaaaagtagaaatactaaacaagcttaaattttagataattaaacatcaattatttgaagtgatgtgataatctatttttttttgtgtttttttaatttcttttctgttattccgaagcttatcagtggagaaaaactagtgaagttgcgaatttcctttaattgcaaaaatgaagaatgttagccacacaccacacttagaagagacaaggagagagacagaaagatgttatcatcacagagtccatagatgtaatagggctcaatatccccttcatcaaattatcacagactcaccaatttcatttctgttccgaccaataaaattttatctttaatttttctccacctgaaaataccaacgtagataattccaccaaaactgaaaattgaactcattggccaccatgggccatggcccccaatttctatttttaacttttagatgtcaaatattaaatttttttaaagtttgttaagatttttaaatcatatacatatacactacgataaaaatcaaattaagtataaatatctctctttttctcatttaatataaatattagttagcattttttttatttcaagaatctttgtatatttttttataaatataaaaggaaaaatattaaattttatatattttttttcataactaatttttaattatgatttggttattggagtttttttagtaattatttatctaagttttcaattagaatatgataaactatttttaaaatttaaattaaaaaatgtatattagttcagaataaatgaataaatttatcttttaaaaaaaaaagttacactcaaaataaagacaagagactcaacttccttatctattccaaagcataataccgaagatttaattagaaaaaatgagacctgagaggttttatatattaataatttggaatgtgatacaaataacattctcaatatatgaaaatattcgatgaatgaaatagatcaaataaagtattcaaaaataatttaaaatagaaaccatatcaaatacaacttacaagttataaattttgaacatattatttttacacccatataattattggtcaagatcatgtgggatcatcagtaccatctttacgtaagtaaaagaccacctcaaatattctggcacttaaaaattctgatattattaccattatgattggttattaagattcaagagcactgaaatgacagtaaccaatgcttgaatttgaataaacgacatgagtctccttcaagacttgtgtggcaaacatatctccttaattctggtggagataggaaaagctgcaataacaacctcaaaatctaagactgagagtaacaacctcaatcttctatgtgtggatgtcaggcttctgtgaccaatccttttgcgcacaagcaactactataagtcagacgtagcctttgatcataagccagctaacactgaatctgcaatcgaagaacaaccttaccctttctctccacaactccaaacaacacttca

SEQ ID NO: 2 - DNA, 2513 bp, Vigna unguiculata
  source 1..2513: mol_type = genomic DNA
agtggctgcacctattctcattttttccgtttcagaaactccgttgtttttctatggctgagggttttagggtaaaaacttgaaaattattaatatgatatcttattagtttgtctcatgtaggaaaactgtaaaaagttgttaattaagcatatctttggtaactaagggtagttagaaacaatttcagaattcatttgaaatgagcaatttggattacaaacaaatcggaaaacgcattttaaattataattttatttgttataattggacaggcataagaaatgtttcacttcagaataggtttttaaattgttatggattacaaactttcaaattacacacttgtctcttagttgctttgtggaacatgtaattcagaataataatttcttaacattactaaaatggtagatggtcattttggatcacgtatattccaaaataaccaccaacaatttcaaactaaaatttattaaatttattaaaaagacttcctttttagtttgactaatgtaactttcttttataaaacactgcagtttgaaatactattttatttgatattttttctgtaactgtacacttatgtataccatattaattcctaagtgatataattaataatgtgctttatagtattacattattacttagtgaatcggatatttcatatgtattaaataccattaacgaaggcgatatcgagttcatattctactagttttttttttttttattttattgatattaaaaatacactttactgatattttaaatatttaaatatcttttaaatatattttaaaatgttaaaattagtaataaatcatatattacgaaagaacaacataaaaataatataaagaaaactagtagaaaatctaaatccggtgatatcaccttttaagtgagttttataaggataaattaaatttaaatgattttttaatatagtaataatagtatcagtgtttttgtaataaagtttcttacgttttttgtgttagagtttactgtatactattaacccaacaaaagcctttttaaataaattattttttaatttctttttatgttactttaggtttgacattactatgaaacatgcaaatttatgtagcataaaaaaacacacgacttaaatttactttagattaggtaatgttcgatataaattgatattagacactacgagatgtaaaaaaaagtctatcacaaataaaaatatatatgaaatctgtattttcttagctcattttagtttatcgtgttatgtattaaacgaaggatttttgtaaagttgaaaaataattgataacagaaagtcaaatttaggataaagtttgagtcacacaagaagaagattgagattatattttcaatgatatatcagtatccttagaattgggatatcgttgaaccttttcagcttaatcttcttcctgagtgactcgacttttgtcttaagtttgacttctttgttcactaactcgtctttaactttatcaaaaattatttctttgatacaaaaaaggttcaaaaatatacatttcattgtaaccttttttattagtagtagaaataaactaagatgagaaaagatgttcgatttttattttaaatgtcatgaaaataaaaaagaaaaacacaatatttttaccactaacatattcttacaatgatacacaataacaaaacaaactaacattcatgcaaaatcaaaactgggtttcccaataaacaataacattcacaaacaaaaatgaaaaattagcaaataaatctcaatgacaaaacaaaattaatgacaacaaaaaaatgaattcccaaaccaccatcattttgtcgtgagagaataaatcctcactctcaaaaatgttttccatgtttttgtgcaattagaaatgaggatgatgtatatttattgtttgaggaaaattatctagtctacattagacaataaaaaatctaacataaattgttttcacttcttacaaaaatactatcatattttttaacatggattttttcactattaaacataaagtagggatataaataactattttttttgttaatatatctgttataataccattcagaaatatttactagatatatgtgtctaaacatgaaagaatatgtgtttgatagtttacatcaattagataaaataaatttatactttccaagttttttataatccaattttattatgtaaaagacagaattttcaagtatttgtgaataaatatttgcagtcattataaaacattatttaaataaagaaaatctaaaccattttttccacgatgaattcaaatacatcagattaaaacacgtgcgtaaaatatgaaatgagattatgaaaaaaaaaaaagaagcttaacctctcactccctcctcaatttttttatttataggacaatatctccctcacactactcacagctcgtgctgtaaagtaagaagaagaagaagaaggtgtgacacacagagaagcagagaaa

SEQ ID NO: 3 - DNA, 2303 bp, Vigna unguiculata
  source 1..2303: mol_type = genomic DNA
actcaatgtgaccctcatcaaaattttggcccaatttaataccaattagttgaaaaatgcgtttgaaacgtggttagtcactgaaatgaccaatttagatatcaatttatagaataacaaattattaattactaaaatagtcagtattatgaataagaaattactaaacattaactactaagatcttgactatccaaaaatttcataaattggtatataaattaatcaccattatttttaataatcaataattttttattttataatttgatatataattgatcaccataatattaatgatgactaacaaactatggtaattaagggaaagattttcttctagtgaaatatggtcattaattaattattacagataatttttttggtagtcaaaacattaacaatttatgtttaatgatttttttatttatagtgactattttagtaactaataattttttatttataataattattttaataactaataaacttttgatttataaattgatatataaattaatcattataaattgatatataaattgattattatattaataactaacaaactaaactatggtaattaaaattcatcattatatttatagaatttggatttcaaattattatgaaagaagatattttgttttataaatctaaataatttattaagttcgttacttaatatgttacttaatatataccgtttaaacaaaaattaaccaatcacacatatttcccgtttttttagttttagtttgttttatttaaacggtaagtcaacatcatcgattgttaagatttattatataaaatattttcttttaatggtaggagggatgattcaaattaactttatttttgtaaaaggaattaataaaagaaaaagaaattaaaatcatgttcatacataatacataaataaacataaaaaatatatgatgtaaaactaccctatatgtatgacttatatcctaattaaaagtaaaaagtaataaaacaaaatcttaactaatagttgccctagaaaggaaaaaatcactgaactgtactgtgcggcgtcgttttggtagccgcataattgaaaggttacctagggcttcgtcacgacattctcgggcccttccgcaccgccagcgaaagcaaaacggatacaactggaactaggtaaaatactgagggtattttcgtcaacacgggaaaaaacttgtagcctataaataaaccctctaaccctcaatccctcacttctcttcactctattgtttgcctttcttctcttgcggctagggttttagcgcagcttcttctaggtatggtctcttataccgatttaagaggtttattccttagattcatgagtatcatattggattgtgttgcctgcagcttatggtttcttgtatctattgctgatgtctttaatttatccaagttttgttgatgaagttttattctttcacatgaattttatgttctttttgatttttgtttgagttgatgattttaatcggttagatgttttcaaaaaaaggattttttttttcggatgcttttctagctttggaacaactggatttggtttgatttttgtggatttgtgtgcttggcatattcaaatccctggatccagatctggtaaacgttttgttgtttggtttgagatttttcttatttctgatgaatagatctgtgaactttttgttttaactgattaaagtcgacgattttatcaggtcttggtgagtatttatgggatgccatgttaaatgtgtggttgtcatgaattatgattgcatgtggtgttgtatcttattttaaacgtgtctataatttgatctgcgtgagtatttagcttgtgttgcgttcgatttgctttaattgattatatccgctagtagtttgcttttggatttgtttttaggtactatctgatatttttgtatgttatgcatgtttttgtttttaggtgtcatccaatttttttatggttatgcatgttttactatgtaaaattttgtgatcgggttttcgatattgtccgataatttgatggttatccatagttttattcttttacgggcattaccccttattttttttttgtggttatgcatgttttcatatgtggaacttatgtggttggtttttagatattacccgatattttctattgctatgcacggtttgttatgcaaatggcgaagctcaccttatttatgttagatgtggctatttattgatttgcattctaactcttgttttgacggttgtttttttgcagatactcaaaggcagtt

SEQ ID NO: 4 - DNA, 2031 bp, Vigna unguiculata
  source 1..2031: mol_type = genomic DNA
attggactgggtctaccgttttgtacttgatcttgtactagagtaaccaactttataaatattttctctttgttgataaatttgataataaaattttgctgatattcttcttaatttctttgatttttcaattatatgtaaaatattaaatatgtaaactaaattttttctaattttattaaataaaacataactaaaaaaacacatatttcctgtcaattttcttttcaaaattttttctcaagaaatacttgcaaactttgttatgaataatgatttgcaagaatttgctaccaattttcttgcaaaatattttgcacaaaaagttttttttgtagaaatttttgtaggaaatgaattttataattttgtaggaaataattacccatgaagaaattgttatccaattaaaattcataggaaaaatagaaaaaaaggtcttttctcgcaaaattttataacaaattttcaaaaaaaaccccacaaaatattaaaaattctagtagtgaactctaactgtattattggaatataattattagatttatgtttgatcttcgcaaaatagggttaaagttggtgtaagaaaaatcaagaattttccttattttaaaaataatgaaaaattttccttctaactttaaaaataatgaagaacttttcatctaattttaaaaataaagaagaatttttcttctaaaacttaaaataaacaaaaattgggactgaattaaacaaaattagtaaaaattgaaactaaattgaaaaaaaattgacaaaaattgagattgaattgaatataagacactaacatgtagtactgacatcgatacatgacactaacaccgatttgacatattatatcaacactgacttgacacgtgtcattgtttatgccgtgtgatataatgttgatttgatatgtggacaactttttttttaattaaaaaaataaaaaaattaaaataactgcaagttgacacatggtgcacactgttcacctcacattaactatttaaacatcgatactgaaaatgatcaaattgacaaaaattaataaaaattgggacattattaaacacccagaaaggagtaataaatttgaacaaaattgacgtgatcaaatttgtatttaatttttttttaactacccagtccaatatagttgtgattttaaaataaactattatattttagataagtaaatcttctaaactatgataattagttgggactaagtaaaatccagattgtttaagagctagaacaaaaataaaataaataagttgtgctacaaaatatcatcttatgatagcaggtaatttggaaggattaactctggaacattcagttggacatgtctggagcacagaaaagcacgtatcacttaaccaaagggaaagcgagaggtgcatatagcagctgaaaaagcaacttctgcgaaactttgtggccattggttctcctttgaaccctttgtggagggggctcattacatcatctcaatcaccattattctagtgtctattaatcaaatgccccattttattttcttgcacaaaatttcataaaatcttttttttttttcatgtgcccattttgttaaccatcattggtttcaacttctagtgtggattagtatgcaaaaaaatctaattgatgaacacatgttcccttatagtcaaacaaacaccagccaaaacttgatccttatgagatgaatgctactagctagttacgtgtggtaaaaatcctcctaacttatggatcaggaaaaggattcaataccaacctcaagttctatggttgtaggtagatgaaaactttcacacactatatggttgccacgtggaagccaagctaatcttctttctgtggatgttatggattcctcaactagtcttgaggatatgaatttctgttcactcaattgcaccattaatataagtccttccctatttgtattttctgctagcctcaaaagacttccgctgtgtttgctttctctgccactctgagtacaatactgtc

SEQ ID NO: 5 - DNA, 1729 bp, Vigna unguiculata
  source 1..1729: mol_type = genomic DNA
tgaccacggaaaccaactccaaaactaaaacaaggttaagaaaggtggtatcgtccacgtctctgctacagcgacgtagatattaccttaaatcctcactctcgcgttttcccttttgttctggcgttggcgttttattaaggcggcgcgtgaagcagggcaaaacagtcagtgggtgtaaaaatatcaatggtgagaggtgagaggtgagaggtgatgaggtgggacccggaggcagacggttacgtgtgacgtttgcggatacaacgtggaaagaatgggcgcgtgagattggatcggatgtagggctagatgaaaaaaatgatcgggtgggagagggtacacgaagtgttaccatagatatttagattgaatttgatattatagatagatagagatagatatctaggaaaaggaggggaggggaagaaggttttagccagcgcctattcctcaccacaattttattccttcctcttcttcgctcactatatattcaataatctctctcttttctcttttcagctactttaggtctttctcttctctctcttccttctttttttttctttcatataattcatcttttatcttcggtttatttttcctttccgctttattctgtgttctgtattgttatcggttatttctgcactgcttccggtgagttctttttgcatccatccttccattccttcattcactgttgtgttactgttactccgtttcgtaccggttgttgcttcaaagctgcttcaattaggttttagaattatccttcttttgtttgtctgtactattagaccactgcgtgcttgtccaattaatgtttttttttgcacgtgctgctgcaatttattgtgcttcttgactcatttattgaggttttcgtttctctcgatttatacgtacataaggaatggttcgcatgttctttgttaagtgttttccgctgtcttattactatctctcgtgatgtaattgcttgatgcgctttttctctattttttgtttgtggatttttataattggcctttccgagctcaaactgtgttgttatgattttgtcgtggttctttgctgctggtgttgatttgttagcactgcattccacttcccacgcttgttactttttacgagttgagtttcctgaagctaaacttctattaaagtgggtgtcactgtggtttccttgttggtttggttttacatacaatagaggaaatagtagattgacaaagttaggaagcggaacatgctatttaattatttatttaagtctcttgttacgttggcactcaacctatctccacgccaagtagtccagatattttgtcctttgttatggatcagttgaagtaactcttgattcttcttagagctatcaatcttaatataggcattgtgcaacttttacttgaacttaattactataacgttggattcatttcaaaattaatgtgatttttagtgtttttttgtcaaactatttagaaaatatatagttcttgtgaacttcaatttaggagattgatcatgtaagtattcatagtctacttttctgaaatatcttttatgttcattacatattatgaaattttgtcctaaatattgatgcttcacgtctgaaacttgtctgcacgcttatctgattgcatttcgtttcttctgccgcatatcatgttttcttgaatgtttcatcaaacatgcaggctgctattgaggaatc

SEQ ID NO: 6 - DNA, 2083 bp, Vigna unguiculata
  source 1..2083: mol_type = genomic DNA
acctctgcaatccccgtttataagccactttctataattattcctttcctcttttatataatatattaaaaattagtaattataccatatcataatttctataaaaaaacactaccaaagaaaattactaattattctaaaaccttaattgcaacataccaaaagtttagtcattaccaaatcttaatacccaaaagctacaagtcatatataatatcaaatacaacaaaaaataaaagagagaaagccgaccccacttttaaccttctttaaatatatatacaaagcagataatttgtttcctttttttctttaatattatataaattactatatgatgaatgtcgtaaaatatcctcctaaatattataacataccataacaccacatttatctactttttcatataactaaaaaaatgaaaactcgagaacgcaaccccaagtcacgctactggaaaaaaaaaaaacataaaccctaatctttcctttgcatcattttacatcttaaaactttaatcaaataaaaacaataacctaactaacttaaatgttattttcaaatataatcttaatccaaaattctaataaaaattcaattttcttttaaataactcaaaaatttaaaaattaaacaaaaacacataatacatgaagcaattggaaacagtggccgccatgagaagaggaacgatggttgtgacttagagaccaccacaacaggtggaaatgcataggagtacgagagagtcgcgatttggatcccaccaaagcacaaagtcaagtaaaaaatcagcattggagattggacatagtcataattgggagcatgacacaagtggcatgatgggatggtggtgaggggacaaggctgggagaatatcgactgagaatgaaaatagggatacaaagactcacacaaaagacatggtgccataacgaagagggagggtgaacttggagtaccatgacgaggctaccagaaaatggaatgcagatgcttggagttcaccaacatgatcatgatcatgtgttacataagagtaaattatgttgttgtaataagacaatttcaccaacgatgaggtgacaatataaaagttaaattatatgtgtgtgacacgttaattatgtttaatgattactaatttttaaagaaaggatgactcaattattatgtttcacaacttaacaaaatataaccataggttcatgataaatgtacttttttaacatgagaaaacacctagttacctattgaagagattaagaatttgataagatattgatcaatcaccataaaataaggataaacctttaattcaaaatttggatgaaaacccgttaaaatcgattttaggatgatattctccctataatcatcttagacatgttgtaaaaaaagacaaaacgctatatctttaaataaaatatcttaaatcaattttcaacattttatatcttaacatatctatagatatataggttgttgattaaggatgagaaaataaccaacccgttataattttgactacctcacggttgactttccaaaaataagataggaaaaaactaacatgtcaacttttttcttatttttaaaattttaaatttctctttaattttgtattttatatattaaaaattttgtatttttaattgattaattaatgtcttgaaataaagttttgtatatatctaaatatttaagtaaaattaaaaagtaaaaagtgacgagagagtttccctagcaccaaattgcactgacaatttatcctttaatgtgaaaaactttaattaaaaaaacaaataattttacttattcaaataaaataaaatacaatataagactggatatggcatgtcgtggcagaatctaggccttacaattcggacgctaaaacggccaaaacccttcctatatgattctagaacaatctctcgcgttatatcttcactaaaccctttcttcatttccttgtaagggttttcgttactctcgctcagactcattcacagatagagataacacataacaaggaagactctcttactcactctcttgtgtgtggagagttcaaagt

SEQ ID NO: 7 - DNA, 1836 bp, Vigna unguiculata
  source 1..1836: mol_type = genomic DNA
aggttctttgagatggcttaccgatctaaaattaatttttataaaagcaaaattagagtcatggtatggataacaataatctaacgaaatactcaaatattttaaattttaacgtaatgaaaatacctttcaaatatttgaatatgcttgtaggaggtaatccgacaaagtagattttttgacaacagtgattacaaaaattaaaagcaaattctttaaatggaaaagtagatatttatcttttatagtataaatctgcttagtaaaattagtaatcacattgtttccattttattatttttcgttttataaagtctcaataagtatttgtaagattgttaaaaacctccaaaaaaaattgtggggatggagacatgagggtagaaaaattgtttgggttaaatggaaaaatttatataaacttaaatacaaaggacaacttggaataaataacattcatttgttgaacaaaactatattaactaaatggaagtgaagattaagcataaatgaaataggattgtgacaagatatcctacaatcaaaatataggtctttgagatgtctaataagatacaatacaaaacattgtacattaattaaggtggtgtaaggacttatatgatgtatgtgactagagggagaagaaaaaaaatggtttgatcataacttgagtggaaagtagagtccagatctaagattaaattttaggaaggtatatgtatagaatcattgtctttacataataggtttcctagagtacatagaaaatcaatttgtttggataaaacaataaggagtttggtgaatgaactaccaatgactaagaatgaaaaatggaatggagaagagattggttttaatgggaattgtctcaatttgaggagtttacacaattgctaaatcaaacacaaatacaaagagaagatgaaacaattttattttttcaaaaaaaaaaaatggcatgatgcaggatcacttcacctataatttcatctatattcccaataaattaaccctattttacaaaaataaaaatcaatcgacccatttttacgattttaacttaaaaagggcctgaaatataaaaaacagtcatgttgcttttgttgtgtatcttgtttagtttttgaaaggtttagcttttatcatttgaatgtaatgagatatcaacgtggtattatttgtttacaaggttaaaacaatccaattactttattattattatcttttgctgataaaaagaaagccttatgatataaattttacgattactttagtgtattaaatttcttttaacatcatcttaaaatttcaattcatcatagtaaacgctaaatagctttagtaacatttatatttattttaagtcactgatatatacacatcatcaaaaaattcaagaactttgttcccaaattttccgtggctaattttcgatactcttatctttatcaaatcgtcacaggtgtcacgttcttcgcacctttcactgacacagaaattcacacccatgcatggcccagcaaggtataccttatccatttgggcctgctccaccgatccaccaagtacagttcgcacgtgtcaactcatgatcctgaaaacgacatcaccactttacacataaaaaaatacaccaacacgtgtccaagcacgtggcagatgcatgtgcaactcacatggcacgtgtcacgcttttgaagagccttgaccagactgttatgctcggtttctcgtataaaatcccatgctaacatgcttcttttcatcaaccagcaccatcacacacaaaagttcttaaaatcacaacctatcacaacaacaagaacaacc

SEQ ID NO: 8 - DNA, 1998 bp, Vigna unguiculata
  source 1..1998: mol_type = genomic DNA
atggtcgaaccgcaagtcatgaaccacgtgtaccattgcctcaagtacaagtggcatgtacgtgacgggccacgtgtcccacgtgcatgggtcacttcttaaaaaaaatcacgattttgctcatcctcattacttggattggacattgggactataacactcgaattaactcttttttttttctgttcaaatcttcaatattatctatttatagtaatttcttaattaataaacacttttttttcattataaaaaccgagtctgcagatggcatgtgaccactttacttgcatatcacatattcattttattatctcaacttttcaaaggcaaattgtaatattttttttagattttttttaaatttaaatatgtatttttttaattttagtttatttttgtatgattttaatttttttaaaattaaaaccagtttattttacacggttgagctaattatatcgcttcaaaatttaaaaatcctgattcggtatctacctctttggaccattctactgattcttttctgattattattattattctttctaagatcaatggacgatgtgttaatcacttttaccaaaaaaaaaagtaaaatataaaaagagaataaaacttattttttatgattatttatcattatcattttatccattatcttcgttaagaaaaaaactgtattattttactaacaataatttaagtgaaaaaagaagaagcaagaactagtttggagtaaaaactgactttttttatattaaattaataaaaagcaggaaaattatctgacttccttaaattttacgaagactttttagttgatttgatttaatttcttttttttctatattttaatgcaagaaaattgtaagaagaaattagaataagattcatgaacgtagaagctagaaagctactagtgtaaacaagcagatctcgtacaatcgggccgtgggcccagtttcattacagtaaggcccgctttccaggcgtcagcaacagggatgaaacagcaatataagtatatataataatcagaataaagattaacatcaggagatattttattcagtaaaagggcaagatatttaagttacaatggcataatggagaatataccgaaaagatagggacataatggaaaagacagaggagattaaaaagagcaggtacttgagaagaaagaaaacgagcgagtgttttagtggctcgggaaggttcgagggccacgtgccagctggttcgggagtccgaagcatgataacgcgactaagaaaacgccacgtcaccactttggggggcaatttaaaaatatcagaaaaatctagagaaggctataaataaagattacagggtgcagcccagagattttctgaatccgctatagccatatcaaaccaacacaaacctcgttacacaaaaaaaacctctcttcactcacaaccaaaacacaaacacactacccaggaaaaagaaaaggataagcctaaaattaaatacaccgcgttttctctactaggttagtagcattggattcttcctcatgttttaatacctctctgtttttagtgcacgctttttaattttttcactgcaaattcttgtgtgcgattttgttttttcattgttgtttttaggtttcctttttggttatttatctgtctgtgtcgtaattccgtgtttttttttttgaaaaggaaaaaactttctgggcaagttttttttttttttttttacatttttatattttggctttagctttatctattacttgggggttacgaaaattcgtttcttggttgaatttattgtggctactgtagtcaaaaggagcgtgttgttgcgaaatcctttttgttattgtccaccttcgtgtgctgcttttgttcttttgagttgctggttactgaattgtttatttatttgttttgaacttcagattgtgaattaagttatcgagttttgctgaggacttagcagtgaagttttgtggac